(19) United States
(12) Patent Application Publication
(10) Pub. No.: US 2024/0390053 A1
(43) Pub. Date: Nov. 28, 2024

(54) DEPLOYABLE STRUCTURES TO PROVIDE ELECTRODES ON THE SURFACE OF AN ENDOSCOPICALLY GUIDED LASER ABLATION CATHETER FOR USE IN ABLATION AND ELECTROPHYSIOLOGICAL MAPPING

(71) Applicant: CARDIOFOCUS, INC., MARLBOROUGH, MA (US)

(72) Inventors: Gerald Melsky, Lexington, MA (US); Omar Colon, Nashua, NH (US); Christopher Brushett, Cambridge, MA (US); Curtis Keohane, Brighton, MA (US); Brian Connor, Bolton, MA (US)

(73) Assignee: CARDIOFOCUS, INC., MARLBOROUGH, MA (US)

(21) Appl. No.: 18/673,932

(22) Filed: May 24, 2024

Related U.S. Application Data
(60) Provisional application No. 63/504,465, filed on May 26, 2023.

(51) Int. Cl. A61B 18/00

(52) U.S. Cl. CPC A61B 18/00; A61B 2018/00077; A61B 2018/00083; A61B 2018/0016; A61B 2018/00184; A61B 2018/0022; A61B 2018/00267; A61B 2018/00577; A61B 2018/00613; A61B 2018/00982

(57) ABSTRACT

An ablation balloon catheter includes an outer catheter shaft and an inflatable balloon coupled at a first end to the outer catheter shaft. The catheter includes a translatable nose tip to which a second end of the inflatable balloon is coupled. The catheter has a first electrode basket having a plurality of first splines and a second electrode basket having a plurality of second splines. The second electrode basket is disposed over the first electrode basket and the plurality of first splines are rotationally offset from the plurality of second splines. One or more of the first splines support one or more electrodes and one or more of the second splines support one or more electrodes. The first and second electrode baskets have a collapsed state when the balloon is deflated and a deployed state when the balloon is inflated.

### CROSS-REFERENCE TO RELATED PATENT APPLICATIONS

This application is based on and claims priority to U.S. Provisional Patent Application No. 63/504,465, filed May 26, 2023, the entire contents of which is incorporated by reference herein as if expressly set forth in its respective entirety herein.

### TECHNICAL FIELD

The present disclosure is directed to ablation of atrial fibrillation and specifically to ablation of atrial fibrillation with a device that includes a deployable structure to provide electrodes on the surface of an endoscopically guided laser ablation catheter for use in ablation and electrophysiological mapping.

### BACKGROUND

Balloon catheters that are configured to perform ablation of atrial fibrillation are well known and are described in Melsky et al U.S. Pat. No. 9,421,066B2 and Melsky et al U.S. Pat. No. 9,033,961B2, each of which is hereby expressly incorporated by reference in its entirety. The aforementioned patents treat atrial fibrillation by using an energy source to create non-electrically conducting lesions in the atrial tissue in such a fashion that a circumferential ring of lesion is created in the region of the left atrium where the pulmonary veins join the atrium. Such circumferential lesions prevent electrical signals originating in the veins from entering the atrium and vice versa. Blocking the passage of such electrical signals can, in most cases, restore sinus rhythm to a previously fibrillating left atrium.

Typically, ablation for atrial fibrillation consists of the steps of introducing an ablation catheter into the left atrium, creating the circumferential lesions around the pulmonary veins and then confirming that the circumferential lesions have been adequately produced so as to actually block electrical signals. This confirmation process generally consists of removing the ablation catheter then introducing a catheter with multiple electrodes which can be placed in a pulmonary vein distal to the circumferential lesion and then using the electrodes to monitor the electrograms originating in the pulmonary veins. When the vein has been electrically isolated from the atrium, the vein is silent with only far-field electrical activity seen in the vein. Occasional spikes within the vein may occur but with no conduction to the rest of the atrium. Pacing the atrium via a catheter with electrodes placed in the coronary sinus can help confirm that only far-field activity and random spikes are seen in the vein.

Now, the aforementioned devices in Melsky et al U.S. Pat. No. 9,421,066B2 and Melsky et al U.S. Pat. No. 9,033,961B2 are effective ablation devices but, as with many other ablation devices, they contain no means for quickly and easily confirming electrical isolation once the ablation of a vein has been completed. It is very desirable to be able to ablate veins and then, without having to exchange catheters, be able to confirm that the ablation has resulted in the desired electrical isolation of the veins. Therefore, one object of the present invention is to provide an ablation device that provides endoscopically guided laser ablation and provides a means to confirm that electrical isolation of the pulmonary veins has been achieved and to perform such confirmation without the need to remove or exchange catheters. Exchanging catheters carries the risk of introducing air into left atrium if performed incorrectly. Air introduction into the left atrium could lead to damage to the brain or heart or of other organs should the air travel into the organs capillary beds and impede blood flow there. For this reason, catheter exchanges are always done slowly and methodically to minimize the risk of air introduction. However, slow and methodical catheter exchanges increase the time to complete an ablation procedure. Prolonged procedures carry other risks to the patient as well as increasing the cost of the procedure so reducing the number of catheter exchanges during a procedure is desirable.

In addition to confirming electrical isolation of the veins has been achieved, the addition of electrodes to the ablation catheters described in Melsky et al U.S. Pat. No. 9,421,066B2 and Melsky et al U.S. Pat. No. 9,033,961B2 would also enable the delivery of ablative energy that requires an electrically conductive pathway from the energy source to the region of ablation. The ablative energy delivered may be either radiofrequency energy or electroporative energy (also called pulsed field ablation energy) or other energy, such as laser or microwave. The ability to deliver these other ablative energy types may be desirable in instances where anatomical considerations favor one type of energy over the other. For example, laser energy is desirable because it creates lesions that penetrate through the full thickness of the atrial wall thus ensuring that the electrical disassociation caused by lesions created using laser energy will be robust and durable. However in circumstances where the esophagus lies against the left atrium in an area that must be ablated, use of electroporative energy in that particular region may be desirable since it has been proposed that electroporative energy creates lesions differentially in cardiac tissue and esophageal tissue thereby opening the possibility that cardiac tissue adjacent to the esophagus can be safely ablated via electroporation without the need to closely monitor the temperature of the esophagus and halt ablation if the esophagus temperature rises too high.

### SUMMARY

In summary, one object of the present disclosure is to provide a means to quickly and easily confirm electrical isolation of pulmonary veins that have been isolated by endoscopically guided laser ablation using devices similar to those described in Melsky et al U.S. Pat. No. 9,421,066B2 and Melsky et al U.S. Pat. No. 9,033,961B2. A further object of the invention is to provide such means in such a manner that no catheter exchanges are required. A further object of the invention is to provide a means to both confirm isolation and to deliver other forms of ablative energy that can be delivered via electrodes which either contact the tissue or are in close proximity to tissue. A further object of the invention is to provide electrodes for either isolation confirmation or ablation that can be visualized endoscopically using the endoscopic apparatus already present in Melsky et al U.S. Pat. No. 9,421,066B2 and Melsky et al U.S. Pat. No. 9,033,961B2.

In one exemplary embodiment, an ablation balloon catheter includes:
an outer catheter shaft;
an inflatable balloon coupled at a first end to the outer catheter shaft;
a translatable nose tip to which a second end of the inflatable balloon is coupled;
a first electrode basket having a plurality of first splines, the first electrode basket being coupled at a first end to the outer catheter shaft and at a second end to the nose tip; and
a second electrode basket having a plurality of second splines, the second electrode basket being coupled at a first end to the outer catheter shaft and at a second end to the nose tip, wherein the second electrode basket is disposed over the first electrode basket and the plurality of first splines are rotationally offset from the plurality of second splines, wherein one or more of the first splines support one or more electrodes and one or more of the second splines support one or more electrodes;
an actuator for axially translating the nose tip to facilitate the first and second electrode baskets moving to a collapsed state when the balloon is deflated;
wherein the plurality of first splines and the plurality of second splines are configured to deploy and radially expand under inflation of the inflatable balloon with the plurality of first splines interposed between the plurality of second splines.

### BRIEF DESCRIPTION OF DRAWING FIGURES

FIG. 1 shows one exemplary device of the present disclosure in a deployed state, deployed over a surface of an inflated balloon of an exemplary balloon catheter;

FIG. 2 shows the device of FIG. 1 in a retracted state ready to be advanced over a deflated balloon of the balloon catheter;

FIG. 3 shows the device of FIG. 1 in a state in which it has been advanced over an inflated balloon of a balloon catheter and it is in a partial state of deployment, such deployment being accomplished by inflation of the balloon;

FIG. 4 shows a PFA catheter mounted basket;

FIG. 5 shows an electrode catheter for use with a balloon catheter;

FIG. 6 shows a dual transeptal/second catheter device that includes the electrode catheter of FIG. 5 disposed over the balloon catheter;

FIG. 7 shows a retractable tine electrode array embodiment;

FIGS. 8A-8C show the various states of the retractable tine electrode array;

FIG. 9 shows a balloon catheter with PFA braided wire mesh electrode array;

FIG. 10 shows a balloon with embedded electrode array;

FIG. 11 shows balloon catheter with micropores with an inner electrode array;

FIG. 12 shows the balloon catheter with micropores with the inner electrode array;

FIG. 13 shows another balloon catheter with micropores and an inner electrode array;

FIG. 14 is a block diagram depicting exemplary components of an endoscope-guided cardiac ablation system according to the invention;

FIG. 15A is a perspective view of a translating tip balloon catheter according to one embodiment and being shown in a collapsed state;

FIG. 15B is a perspective view of the translating tip balloon catheter of FIG. 15A in an expanded state;

FIG. 16A is a perspective view of a handle suitable for use with the translating tip balloon catheter of FIG. 15A and being shown in an extended state; and

FIG. 16B is a perspective view of the handle of FIG. 16A in a retracted state.

### DETAILED DESCRIPTION OF CERTAIN EMBODIMENTS

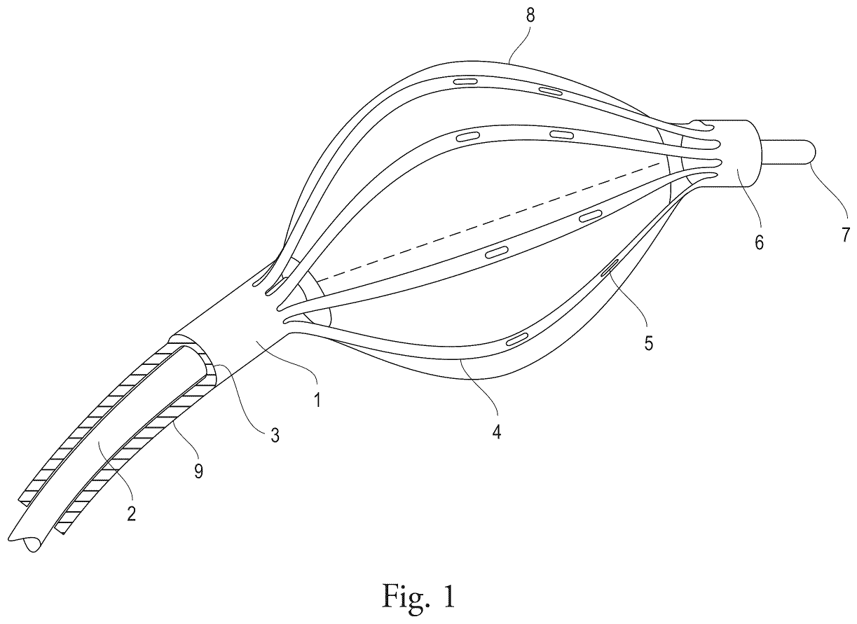

FIG. 1 shows an exemplary balloon catheter, such as the one described in Melsky et al U.S. Pat. No. 9,421,066B2 and Melsky et al U.S. Pat. No. 9,033,961B2, each of which has been incorporated by reference.

### Exemplary Ablation System

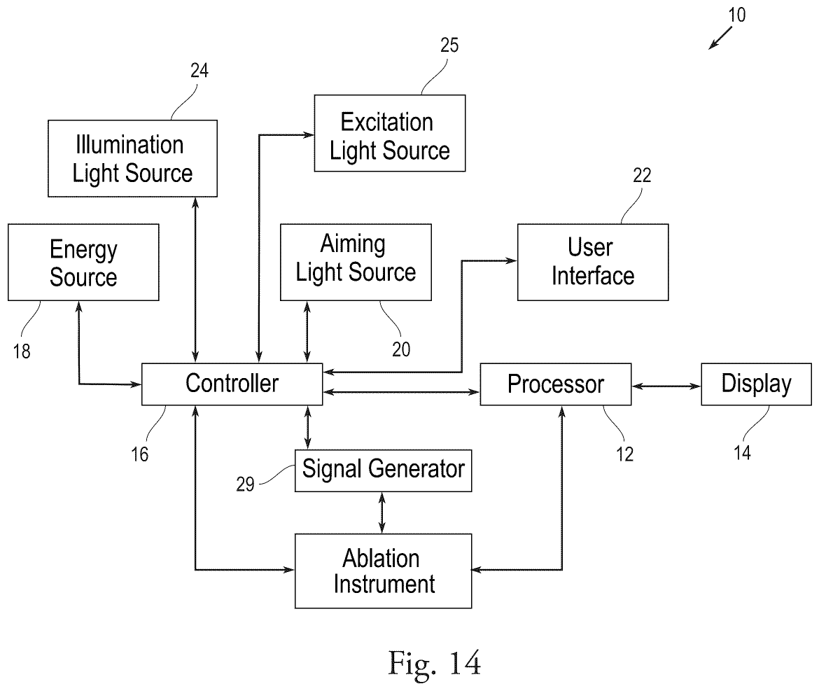

FIG. 14 is an exemplary schematic block diagram illustrating one ablation/endoscopic system in accordance with the invention, designated generally by reference numeral 10. Ablation system 10 preferably includes a treatment ablation instrument, such as one of the ones described herein, preferably including an endoscope and ablation device as discussed below.

The ablator system 10 further preferably includes an aiming light source 20 and an illumination light source 24. A processor 12 designed to accept input and output data from the connected instruments, a display 14, and a controller 16 and process that data into visual information.

As will also be appreciated from the below discussion, an endoscope is preferably provided in ablation instrument 100 and has the capability of capturing both live images and recording still images. An illumination light 24 is used to provide operating light to the treatment site. The illumination light is of a frequency that allows the user to differentiate between different tissues present at the operating site. An aiming light source 20 is used to visualize the location where energy will be delivered by the ablation instrument 100 to tissue. It is envisioned that the aiming light 20 will be of a wavelength that can be recorded by an image capture device and visible on a display.

The processor 12 can be designed to process live visual data as well as data from the ablation instrument controllers and display. The processor 12 is configured execute a series of software and/or hardware modules configured to interpret, manipulate and record visual information received from the treatment site. The processor 12 can further be configured to manipulate and provide illustrative and graphical overlays and composite or hybrid visual data to the display device.

As seen in FIG. 14, the system 10 further includes the controller 16, an energy source 18, the aiming light source 20 and a user interface 22. Controller 16 is preferably configured to control the output of the energy source 18 and the illumination and excitation sources 24 and 25 of an energy transmitter, as well as being configured to determine the distance and movement of an energy transmitter relative to tissue at an ablation treatment site (as discussed further below). As will also be appreciated from the below discussion, an endoscope is preferably supported by the ablation instrument and captures images that can be processed by the processor 12 to determine whether sufficient ablative energy deliveries have been directed to a specific area of a treatment site. Data obtained from the endoscope includes real-time video or still images of the treatment site as seen from the ablation instrument. As discussed herein, these images/videos can be stored in memory for later use.

The aiming light source 20 is used to visualize the treatment site location where energy will be delivered by the ablation instrument to the target tissue. Preferably, the aiming light source 20 outputs light in a visible region of the electromagnetic spectrum. If a suitable ablation path is seen by the user, the controller 16 can transmit radiant energy, via energy source 18, from the ablation instrument to a target tissue site to effect ablation by lesions. It is to be appreciated that the term “radiant energy” as used herein is intended to encompass energy sources that do not rely primarily on conductive or convective heat transfer. Such sources include, but are not limited to, acoustic, laser, electroporative energy, and electromagnetic radiation sources and, more specifically, include microwave, x-ray, gamma-ray, ultrasonic and radiant light sources. Additionally, the term “light” as used herein is intended to encompass electromagnetic radiation including, but not limited to, visible light, infrared and ultraviolet radiation.

The illumination light source 24 is a light source used to provide proper illumination to the treatment site. The illuminate is configured so that natural biological tones and hues can be easily identifiable by an operator.

The controller 16 can provide the user with the ability to control the function of the aiming light source, the user input devices, and the ablation instrument. The controller 16 serves as the primary control interface for the ablation system. Through the controller 16, the user can turn on and off both the aiming and illumination lights 20, 24. Furthermore the controller 16 possesses the ability to change the illumination and aiming light intensity. The ability to switch user interfaces or display devices is also envisioned. Additionally, the controller 16 gives access to the ablation instrument, including control over the intensity of the discharge, duration and location of ablative energy discharges. The controller 16 can further provide a safety shutoff to the system in the event that a clear transmission pathway between the radiant energy source and the target tissue is lost during energy delivery (e.g., see commonly owned U.S. patent application Ser. No. 12/896,010, filed Oct. 1, 2010, which is hereby incorporated by reference in its entirety).

The controller can be a separate microprocessor based control interface hardware or it can be a portion of a configured as a module operating through a processor based computer system configured to accept and control inputs from various physical devices.

### Pulsed Electric Field Ablative Energy

While the technical field of pulsed electric fields for tissue therapeutics continues to evolve, it is generally understood that application of brief high DC voltages to tissue may generate locally high electric fields typically in the range of hundreds of volts per centimeter that disrupt cell membranes by generating pores in the cell membrane. While the precise mechanism of this electrically-driven pore generation or electroporation continues to be studied, it is thought that the application of relatively brief and large electric fields generates instabilities in the lipid bilayers in cell membranes, causing the occurrence of a distribution of local gaps or pores in the cell membrane. This electroporation may be irreversible if the applied electric field at the membrane is larger than a threshold value such that the pores do not close and remain open, thereby permitting exchange of biomolecular material across the membrane leading to necrosis and/or apoptosis (cell death). Subsequently, the surrounding tissue may heal naturally.

Generally, a system, such as the ones described herein, for delivering a pulse waveform to tissue includes a signal generator configured for generating a pulse waveform and an ablation device coupled to the signal generator and configured to receive the pulse waveform. In some embodiments, the ablation device is configured to generate an electric field intensity of between about 200 V/cm and about 1500 V/cm. Accordingly, a system for ablating tissue described herein can include a signal generator and an ablation device having one or more electrodes and an expandable/inflatable member (e.g., balloon) for the selective and rapid application of DC voltage to drive electroporation.

In some embodiments, the voltage pulse waveforms disclosed herein may be hierarchical and have a nested structure.

An irreversible electroporation system as described herein may include a signal generator and a processor configured to apply one or more voltage pulse waveforms to a set of electrodes to deliver energy to a region of interest. In order to deliver the pulse waveforms generated by the signal generator, one or more electrodes of the ablation device may have an insulated electrical lead configured for sustaining a voltage potential of at least about 2500 V without dielectric breakdown of its corresponding insulation at least in one embodiment. In some embodiments, at least some of the electrodes may be independently addressable such that each electrode may be controlled (e.g., deliver energy) independently of any other electrode of the device.

As shown in FIG. 14, the system can include a signal generator 29 that is configured to generate pulse waveforms for irreversible electroporation of tissue, such as, for example, a pulmonary vein. For example, the signal generator 29 can be a voltage pulse waveform generator and be configured to deliver a pulse waveform to one of the ablation devices (ablation instruments) described herein. The processor 12 can incorporate data received from memory to determine the parameters of the pulse waveform to be generated by the signal generator 29, while some parameters such as voltage can be input by a user. The memory can further store instructions to cause the signal generator 29 to execute modules, processes and/or functions associated with the system, such as pulse waveform. For example, the memory can be configured to store pulse waveform for pulse waveform generation.

Some embodiments are directed to pulsed high voltage waveforms together with a sequenced delivery scheme for delivering energy to tissue via sets of electrodes. The signal generator and the processor are capable of being configured to apply pulsed voltage waveforms to a selected plurality or a subset of electrodes of an ablation device.

In one application, a pulsed voltage waveform can be in the form of a sequence of double pulses, with each pulse, such as the pulse being associated with a pulse width or duration. The pulse width/duration can be about 0.5 microseconds, about 1 microsecond, about 5 microseconds, about 10 microseconds, about 25 microseconds, about 50 microseconds, about 100 microseconds, about 125 microseconds, about 140 microseconds, about 150 microseconds, including all values and sub-ranges in between. The pulsed waveform can be defined by a set of monophasic pulses where the polarities of all the pulses are the same (e.g., all positive, as measured from a zero baseline). In some embodiments, such as for irreversible electroporation applications, the height of each pulse or the voltage amplitude of the pulse can be in the range from about 400 volts, about 1,000 volts, about 5,000 volts, about 10,000 volts, about 15,000 volts (e.g., in one application a maximum amplitude of 2500 volts is used), including all values and sub ranges in between. The pulse is separated from a neighboring pulse by a time interval, also sometimes referred to as a first time interval. As examples, the first time interval can be about 1 microsecond, about 50 microseconds, about 100 microseconds, about 200 microseconds, about 500 microseconds, about 800 microseconds, about 1 millisecond including all values and sub ranges in between, in order to generate irreversible electroporation. It will be appreciated that the aforementioned values are only exemplary in nature and are not limiting of the scope of the present invention since values outside the aforementioned ranges can exist for other applications.

### Exemplary Ablation Catheter

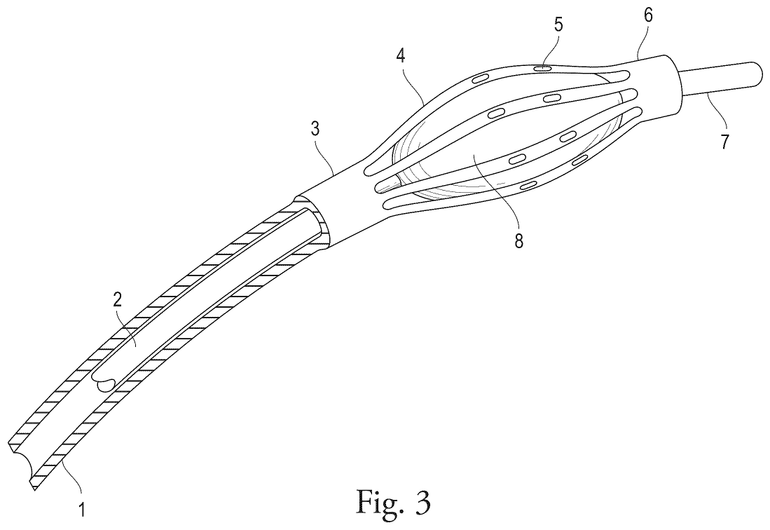

As shown in FIGS. 1-3, one exemplary ablation device is directed to a generally flexible and elongate structure 1, that is slidably disposed over an elongate shaft 2 of a balloon ablation catheter. The elongate structure 1 can be considered to be a sleeve that is longitudinally displaceable over the balloon catheter. While the term “elongate structure” is used herein, it will be understood that the term “sleeve” can be interchangeably used therewith. As described herein, the elongate structure 1 can be moved along the balloon catheter so as to cover different regions of the balloon catheter. As described herein, the elongate structure 1 is configured to respond to the movements of the balloon ablation catheter and more particularly, to the expansion and contraction of the balloon when the elongated structure at least partially covers the balloon.

The elongate structure 1 generally has several different portions including a proximal portion and a distal portion. The proximal portion of the elongate structure 1 comprises a first tubular shaped portion 3 as shown in FIG. 1. This proximal region is spaced back from the distal end at a distance of 2 cm to 4 cm; however, this is merely one exemplary value and not limiting of the scope of the present invention. The first tubular shaped portion 3 is configured such that the shaft 2 of the balloon ablation catheter passes through a lumen of the first tubular shaped portion 3. In other words, the first tubular shaped portion 3 completely surrounds the catheter shaft 2 in at least one region of the first tubular shaped portion 3.

The first tubular shaped portion 3 can be formed of a flexible material.

The distal portion of the elongate structure 1 multifurcates into two or more but preferably six or more branches 4, which are also flexible. Each branch 4 contains one or more electrodes 5 on their outward facing surface. Each electrode 5 is connected to an insulated conductor wire imbedded in the body of the elongate flexible structure 1 but such conductor wires or the like are not shown in FIG. 1. For example, the structure 1 can be overmolded over the conductor wires. As shown, when multiple electrodes 5 are used for each branch 4, the electrodes 5 are spaced longitudinally apart along the respective branch 4. It will also be appreciated that the electrodes 5 can be of the same type or can be different types. In other words, the electrodes 5 can be of different sizes and/or different shapes. The arrangement of the electrodes 5 can be of an asymmetric nature in that the electrodes 5 can be focused on one or more regions of the branches 4. For example, the electrodes 5 can be more centrally located and distally located along the branches 4 as opposed to be located proximally.

The branches 4 can thus be circumferentially spaced apart from one another and extend circumferentially about the balloon. It is also possible for the branches 4 to be designed to have an asymmetric appearance in that instead of having a symmetric angular displacement between the branches 4, an asymmetric arrangement can be provided. In other words, within one half of the elongate structure 1, the branches 4 can have one type of angular displacement and within the other half, a different angular displacement can be provided. In other words, there can be more branches 4 in one half of the structure 1 compared to the other half of the structure 1. For example, the first circumferential half can have a first number of electrodes, while the second circumferential half can have a second number of electrodes that can be different than the first number.

As shown, each branch 4 has a first end (proximal end) and an opposing second end (distal end). The first ends of the branches 4 are attached to the first tubular shaped portion 3 and in one embodiment, the branches 4 are formed integral with the first tubular shaped portion 3.

The multiple flexible branches 4 rejoin at their second ends to again form a second tubular structure 6 at the distal end of the elongate structure 1. The second tubular structure 6 encircles, in a slidable manner (both axially and rotationally), a distal tip 7 of the balloon ablation catheter.

In general, the multifurcations (branches 4) form an expandable cage like structure which circumferentially surrounds the inflated balloon 8 when the elongated structure is positioned over at least a portion of the balloon. The proximal portion of the elongate structure 1 can maintain a tubular shape proximally from the multifurcations (branches 4) on back or, alternatively, the proximal portion of the elongate structure 1 can consist of only a partial circumferential portion of a tube as shown at 9 and thereby be more flexible and occupy less volume than if it were entirely tubular. Shaft 2 can be visible between portions of the elongate structure 1.

It will be appreciated that the present device 1 is preferably formed as a single elongate structure in which the tubular portions 3, 6 and branches 4 located therebetween are formed as a single unitary part (e.g., molded part).

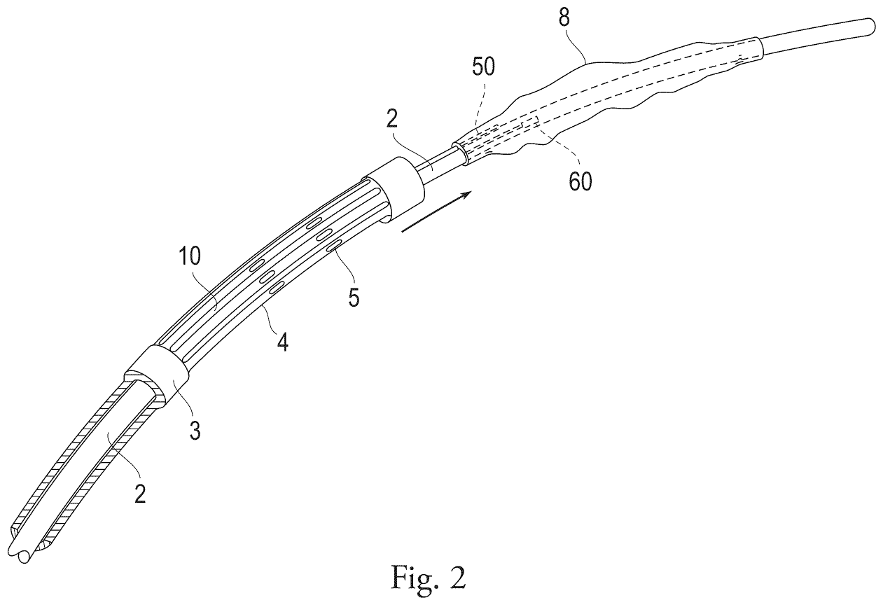

FIG. 2 shows travel of the elongate structure 1 over the balloon catheter. More particularly, the first tubular shaped portion 3 and the branches 4 are shown in their relaxed state. This represents the normal, at rest state of the elongate structure 1. In this state it is clear to see how such a structure can be produced by creating a series of longitudinal slits 10 in a generally thin flat material that has been formed into a tubular shape. In other words, the branches 4 are formed by incorporating longitudinal slits in the structure 1 so as to define one branch between two adjacent slits. A suitable thin flat material would be polyimide film such as is commonly used to produce flexible printed circuits or flex-circuits. It will be appreciated that other materials are equally possible.

FIGS. 1 and 2 together illustrate how the present device accomplishes the objectives of providing a means to allow for pulmonary vein isolation using an endoscopically guided balloon catheter and to additionally provide a means to confirm electrical isolation of the vein without the need to exchange catheters as required in the prior art. As discussed in more detail below, the inner surface of the tubular structure can contain marks on the inside surface that are visible to the endoscope for indicating the location of the electrodes.

There can thus be two defined stages of operation including a first stage which is an ablation stage of the procedure in which the elongate structure 1 is not used. During this ablation stage, the elongate structure 1 resides, as shown in FIG. 2, proximal to the balloon of the balloon catheter and in a collapsed state closely surrounding the shaft 2 of the balloon catheter. As shown in this stage and state, the entire elongate structure 1 is displaced from and located proximal to the balloon of the balloon catheter. The distal second tubular shaft portion 6 is thus located proximal to the balloon.

In this state (first stage), the present elongate structure 1 allows for the balloon of the ablation catheter to be inflated and placed in a pulmonary vein while not being encumbered by the elongate structure 1. The vein may be visualized endoscopically by the ablation catheter and laser energy may be delivered to the vein without regard to the invention. In other words, as in Applicant's previous ablation catheter designs, energy from a movable energy emitter 0 (FIG. 2) that resides within the balloon passes through the balloon to the target site without any impediment from the elongate structure 1 due to the elongate structure 1 being spaced from and not in contact with the inflated operating area of the balloon.

This would not be the case if electrodes (such as electrodes 5) had been placed directly on the surface of the balloon since such electrodes would block both the laser energy and endoscopic visualization over the portion of the balloon on which such electrodes resided.

Once ablation (the first stage) of a vein has been accomplished, the balloon of the ablation catheter is deflated but the elongate structure 1 of the ablation catheter is not repositioned relative to the vein. With the ablation catheter structure stationary relative to the ablated vein, the elongate structure 1 is advanced distally over the deflated balloon. The balloon is then re-inflated and such re-inflation expands the branches (multifurcations) 4 of the elongate structure 1 and forces at least some number of the electrodes 5 into contact with the lumen of the vein. Said electrodes 5 can now be used to confirm electrical isolation by connecting the conductor wires connected to the electrodes 5 and extending proximally along the proximal portion of the elongate structure 1 until they are present outside of the patient's body, to know devices which are capable of amplifying and displaying the electrical activity emanating from the tissue in contact with the electrodes 5.

It should also be noted that the electrodes 5, when in this state of contact with the pulmonary vein tissue (or other target tissue) are also capable of delivering ablative energy such as radiofrequency energy or electroporative energy or microwave energy by connecting a source of such energy to the conductor wire attached to the electrodes. It should be also noted that the positions of the electrodes are visible to the endoscope 50 (FIG. 2) which resides inside the balloon of the ablation catheter. This visibility is accomplished by either making the multifurcations 4 out of a transparent material or by creating marks on the inner surfaces of the multifurcations directly adjacent to the position of the electrodes. Such visualization of the electrode position endoscopically enables a visual assessment of the condition of contact between electrodes and tissue. For example, a given electrode may be in firm contact with the vein tissue throughout the entire cardiac cycle. Alternatively, the electrode 5 can be in contact with tissue during a portion of the cardiac cycle and during the other portion of the cycle, the electrode 5 may not be in contact with tissue but it is in contact with blood instead or the electrode may not be in contact with tissue during any part of the cardiac cycle. Such visual assessments of the nature of the contact between tissue and the electrodes in not currently available in any know devices. Such assessment is valuable in aiding interpretation of the electrograms measured by the electrodes. Further, if the electrodes are to be used for the purposes of applying radiofrequency or electroporative or microwave ablation energy, such visual information about the degree of tissue contact can be used to determine which of the several electrodes are suitable to deliver ablative energy by virtue of the degree of tissue contact they afford. Also, the endoscopic view can be used to guide the repositioning of the balloon in the vein in order to improve the contact between electrodes and the vein tissue if deemed necessary for a better assessment of the electrical activity in the vein or for better electrode contact to enable ablation via radiofrequency or electroporative energy application.

### Sliding Action of Elongate Structure 1

As discussed herein, the elongate structure 1 is configured to move longitudinally along the balloon catheter as illustrated in FIGS. 1-3. It is also to have rotational movement relative to the balloon. The elongate structure 1 can be moved manually as by grasping one end (such as the first tubular portion 3) of the elongate structure 1 and the moving the entire structure 1 longitudinally in a distal or proximal direction. Alternatively, to move the elongate structure 1 in the proximal direction, the first tubular portion 3 can be grasped and pulled in the proximal direction. Preferably, the first tubular portion 3 extend proximally to a point where it exits the body and is available to be grasped directly by the user. To assist the user in moving the structure 1, the most proximal end of the structure 1 can have a grasp feature, such as an enlarged ring section or the like at the proximal end of the first tubular portion 3. Alternatively, surface texture or the like can be provided to one or more regions of the first tubular portion 3.

When the elongate structure 1 is retracted and moved proximally, it can enter into a lumen formed in the catheter structure or into a lumen in a guiding sheath or deflectable sheath commonly employed in atrial ablation procedures, through which the balloon catheter and tubular structure would be passed. This to say that the tubular structure can be slid so that it is retracted into the catheter shaft or into a guiding or deflectable sheath and this retraction will cause the elongate structure 1 to collapse and be removed from surrounding relationship around the balloon. The retraction of the structure 1 within the lumen of the catheter shaft causes the collapsing of branches to a compact state. It is noted that when the tubular structure is retracted into a guiding or deflectable sheath the multifurcations of the tubular structure are supported and prevented from expanding or deflecting outwardly by the inner surface of such sheath and are also prevented from deflecting inwardly by the shaft of the balloon catheter. In such a state the tubular structure is constrained from expanding or contracting and is therefore more easily repositioned relative to the balloon catheter. In the case of a device were the only ablative energy employed is delivered via the electrodes, the elongate structure would not necessarily need to be retracted to a position fully proximal of the balloon. In other words, the elongate structure 1 is movable between a multitude of positions with one position being a position in which at least some electrodes at least partially cover the balloon.

### Controllable Electrodes

The overall ablation system described herein that includes the elongate structure 1 and the ablation balloon catheter can communicate over a network to the various machines that are configured to send and receive content, data, as well as instructions that, when executed, enable operation of the various connected components/mechanisms. The content and data can include information in a variety of forms, including, as non-limiting examples, text, audio, images, and video, and can include embedded information such as links to other resources on the network, metadata, and/or machine executable instructions. Each computing device can be of conventional construction, and while discussion is made in regard to servers that provide different content and services to other devices, such as mobile computing devices, one or more of the server computing devices can comprise the same machine or can be spread across several machines in large scale implementations, as understood by persons having ordinary skill in the art. In relevant part, each computer server has one or more processors, a computer-readable memory that stores code that configures the processor to perform at least one function, and a communication port for connecting to the network. The code can comprise one or more programs, libraries, functions or routines which, for purposes of this specification, can be described in terms of a plurality of modules, residing in a representative code/instructions storage, that implement different parts of the process described herein. As described herein, each of the robotic devices (tools) has a controller (processor) and thus, comprises one form of the above-described computing device.

Further, computer programs (also referred to herein, generally, as computer control logic or computer readable program code), such as imaging or measurement software, can be stored in a main and/or secondary memory and implemented by one or more processors (controllers, or the like) to cause the one or more processors to perform the functions of the invention as described herein. In this document, the terms “memory,” “machine readable medium,” “computer program medium” and “computer usable medium” are used to generally refer to media such as a random access memory (RAM); a read only memory (ROM); a removable storage unit (e.g., a magnetic or optical disc, flash memory device, or the like); a hard disk; or the like. It should be understood that, for mobile computing devices (e.g., tablet), computer programs such as imaging software can be in the form of an app executed on the mobile computing device.

The system can include a graphical user interface (GUI) that can be provided to allow for remote control over the system. As is known, the GUI is a system of interactive visual components for computer software. A GUI displays objects that convey information and represent actions that can be taken by the user. The objects change color, size, or visibility when the user interacts with them. GUI objects include icons, cursors, and buttons. These graphical elements are sometimes enhanced with sounds, or visual effects like transparency and drop shadows.

The graphical user interface typically includes a display, such as a touch screen display to allow user input to be registered and then steps are taken by the main controller (main processor).

In one exemplary embodiment, the main controller can be used to control the operation of the electrodes 5. In other words, select electrodes 5 can be operated (activated) at a given time using the main controller. Those electrodes 5 that are activated are supplied with ablative energy, while those electrodes 5 that are not activated are not supplied with ablative energy. As mentioned, the electrodes 5 can be wired to an electrical connector that is itself connected to a terminal (console) or the like (e.g., an outlet or plug thereof), thereby providing power to the electrodes 5.

Depending upon certain parameters, such as the location of the balloon catheter in the body, certain electrodes 5 can be activated and turned on, while certain electrodes 5 can be turned off and not activated. For example, if the balloon catheter and tubular structure are contacting certain tissue and such contact with tissue is being visualized by an endoscope inside the balloon, the user may want only those electrodes 5 that are in contact with tissue to receive ablative energy and therefore, based on endoscopic guidance or the like, the operator can strategically select which branches 4 and electrodes 5 that are to be activated.

The master controller can communicate with a display on which images and data can be displayed.

A touch screen or the like can be used to select the branches 4 and electrodes 5 that are to be activated (energized). For example, a graphic image of the elongate structure 1 and more specifically, a graphic image of the branches 4 and the electrodes 5, can be displayed to the operator and then the operator can select those branches 4/electrodes 5 to be activated. When a touch screen is used, the operator can simply highlight and select with a finger those branches 4/electrodes 5 that are to be activated. It will also be appreciated that AI based software can be used to determine and then recommend to the user which electrodes should be activated based on the determination that those electrodes are in contact with tissue.

### PFA Catheter Mounted Basket

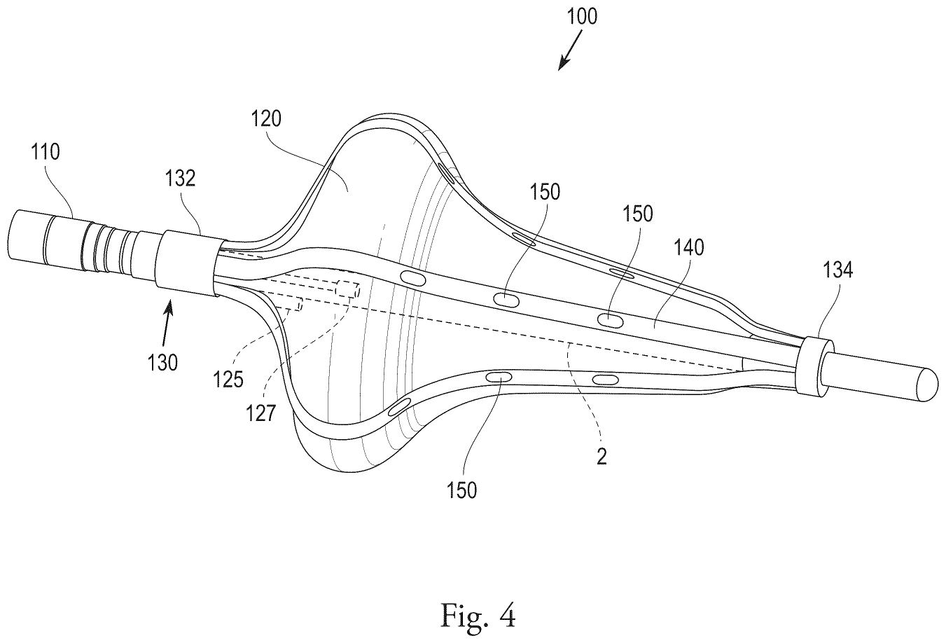

FIG. 4 illustrates a balloon catheter 100 that includes a main catheter shaft 110 that has a distal end. It will also be appreciated that the balloon catheter 100 typically includes more than one shaft and often includes an inner catheter shaft and an outer catheter shaft or can otherwise include multiple concentric tubular structures. An inflatable balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

FIG. 4 also shows an inner shaft 115 along with an endoscope 125. The endoscope 125 extends along the exterior of the inner shaft 115 and is typically located at one end of the balloon is forward looking in that it looks forward toward the other end of the balloon.

The inflatable balloon 120 is preferably a compliant balloon.

The inflatable balloon 120 also includes an endoscope 125 that is located within the compliant balloon. The endoscope allows the operator of the catheter to visualize the balloon surface and thereby aim the laser energy to those portions of the balloon surface which contact the atrial tissue it is desired to treat with the laser energy. Such a system is described in Melsky et al. (U.S. Pat. No. 9,421,066 (the '066 patent) and Melsky et al. (U.S. Pat. No. 9,033,961 (the '961 patent), each of which is incorporated by reference in its entirety. The endoscope is at a location that is proximal to the location at which the energy is delivered to the tissue to allow the user to view the delivery of the energy and the resultant tissue lesion(s). The endoscope can be one of the ones described herein and also one that is described in any one of the documents incorporated by reference herein.

In FIG. 4, an energy emitter 127 is illustrated; however, it will be appreciated that in the embodiment in which the electrode array is intended to remain in a position surrounding the inflatable balloon 120, then the energy emitter 127 can be eliminated or is present but never used. In the event that the electrode array can be displaced off of the balloon, then the energy emitter 127 can be used.

The endoscope 125 is forward-facing and is disposed adjacent to one of the catheter shafts, such as a central tubing typically formed of a transparent polymer material. As used herein, the term forward-facing refers to the view of the endoscope in a distal direction relative to the catheter body. Similarly, the term side-facing refers to the view of the endoscope in a direction that is radially outward from a side of the catheter body.

The endoscope 125 can be a fiber optic endoscope that is inserted through a lumen of the catheter and located within a proximal region of the inflatable balloon 120.

In another embodiment, the ablation catheter 100 includes first and second imaging devices for providing direct visualization of the region to be treated, with the first imaging device being fixed relative to the catheter body. The first and second imaging devices can be in the form of first and second imaging chip endoscopes. Details of the first and second imaging chip endoscopes are described in U.S. patent application Ser. No. 17/524,472, which is expressly incorporated herein by reference in its entirety.

The balloon catheter 100 includes an expandable basket 130 that surrounds the inflatable balloon 120 and is configured to expand upon expansion (inflation) of the inflatable balloon 120 and similarly, is configured to contract upon deflation and contraction of the inflatable balloon 120. The expandable basket 130 has a first collar (first ring) 132 at a first (proximal) end of the expandable basket 130 and a second collar (second ring) 134 at a second (distal) end of the expandable basket 130. The first and second collars 132, 134 have annular shapes and can thus have a continuous ring shape. The size of the two collars 132, 134 can be different from one another with the first collar 132 in the illustrated embodiment being larger than the second collar 134. The two collars 132, 134 are sized and configured to fixedly couple the expandable basket 130 to the main catheter shaft 110 (or one or more other catheter shafts) with the inflatable balloon 120 being located between the two collars 132, 134. The first collar 132 is thus preferably located proximal to the inflatable balloon 120, while the second collar 134 is located distal to the inflatable balloon 120.

The expandable basket 130 includes a plurality of splines 140 that are attached at one end to the first collar 132 and at the other end to the second collar 134. The plurality of splines 140 extend longitudinally along a length of the inflatable balloon 120. The plurality of splines 140 are circumferentially offset from one another with open spaces formed between adjacent splines 140. The splines 140 are constructed to expand and contract under action of the underlying inflatable balloon 120. In particular, when the inflatable balloon 120 expands under inflation, the splines 140 expand outwardly and conversely, when the inflatable balloon 120 contracts under deflation, the splines 140 contract inwardly. The splines 140 thus conform to the shape of the inflatable balloon 120.

Each spline 140 carries one or more electrodes 150. For example, each spline 140 can include a plurality of electrodes 150 that can be described as being an electrode array. In the illustrated embodiment, there are three electrodes 150 located along the length of the spline 140. The electrodes 150 are spaced longitudinally along the spline (in series). The electrodes 150 are thus spaced apart from one another a predefined set distance. The locations of the splines 140 along the spline 140 are selected so as to centrally position the electrodes 150 relative to the inflatable balloon 120 since when the inflatable balloon 120 is inflated, the electrodes 150 are, as discussed herein, for placement against the target tissue to be ablated using PFA technique.

The electrodes 150 that define the electrode can be the same electrode type or they can be different. For example, the shapes and sizes of the electrodes 150 can be the same as shown. The material of the expandable basket 130 is not elastic in that the splines do not stretch elastically in a longitudinal direction but can expand and contract with the underlying inflatable balloon 120. Thus, the longitudinal spacing between the electrodes 150 does not change when the expandable basket 130 moves between the expanded position and the retracted position. Instead, it is a fixed distance which is important and this information is used during the visualization and ablation process in order to form the desired lesion as discussed herein.

Compared to the embodiment of FIGS. 1-3, FIG. 4 shows a product in which the expandable basket 130 is fixed at least in one embodiment.

In yet another aspect of the present disclosure, the system can include electrode markers which mark the location of the electrodes 150 along the splines. In particular, the electrodes 150 are located on the outer surface of the splines 140 and the splines are typically formed of a non-transmissive material and therefore, the electrodes 150 are not visible in the live endoscope image. Since the splines 140 are typically formed of an opaque material, the electrodes 150 cannot be seen since the endoscope 125 only sees the inner surface of the splines 140. In order for the locations of the electrodes 150 to be determined during visualization (i.e., use of the endoscope 125), markers can be provided along the inner surface of the spline 140. Each marker is located on the inner surface of the spline 140 directly opposite the location of the electrode 150 to mark the location of the electrode 150. The markers are visually identifiable in the live endoscopic image and therefore can be in the form of visual indicia formed along the inner surface of the spline 140. For example, the visual indicia can be in the form of numbers and/or text indicia. In addition, the visual indicia is selected such that one electrode can be differentiated from another electrode. For example, each spline can be numbered, such as spline 1, and then each electrode 150 can be lettered, such as A, B, C, etc. Thus, in the illustrated embodiment, the most distal electrode of spline 1 can be identified by marker 1A, the middle electrode can be identified by marker 1B and the most proximal electrode can be identified by marker 1C. Similarly, for the adjacent spline 2, the markers can be 2A, 2B, and 2C. It will be appreciated that there are many different ways to visually identify one electrode on one spline from another electrode on another spline.

For example, color can be used to identify one spline 140 from the other ones. For example, the letters A, B, C or numbers 1, 2 and 3 can be in one color for one spline and another color for another spline. Symbols can also be used as the markers.

It will be appreciated that not all of the electrodes 150 are visible in the live endoscopic image since not all of the electrodes are in the desired contact with tissue at the target site and therefore it is important to understand which electrodes are visible in the live endoscopic image and in contact with tissue so that these electrodes can be actuated (activated).

The movement of the expandable basket 130 and the inflatable balloon 120 can vary depending on the embodiment. For example, in one embodiment, the expandable basket 130 and the inflatable balloon 120 can move together, while in another embodiment, the basket 130 can move independently from the balloon 120. For example, the basket 130 can be fixed in the rotational direction but can move in the axial (longitudinal direction) or in another embodiment it can be fixed.

The movement of the expandable basket 130 relative to the catheter body and the inflatable balloon 120 can be either an automated process as by using an electronic controller or it can be a manual process that occurs under action of the user. The controls permit the desired movements in rotational and/or longitudinal directions.

Delivery of energy and electrode selection: In one embodiment, energy is delivered to two or more electrodes 150 that are located along the same spline 140. In this embodiment, since the distance between the electrodes 150 on one spline 140 is fixed and does not change based on the expansion of the basket. This allows the PFA dosing to be selected since the distance between the electrodes 150 to be activated is known. In another embodiment, energy is delivered between two electrodes 150 that are not located along the same spline 140 but rather are located along adjacent splines 140. In this case, the distance between the splines 140 does change depending on the degree of basket expansion. For example, the greater the degree of basket expansion, the greater the distance between the splines 140 and thus, the greater the distance between the electrodes 150. When the electrode spacing remains fixed, there is a greater degree of dosing predictability.

In view of the visualization information and the location of and spacing between the electrodes that are to be actuated to cause lesion formation, the (PFA) dosing is selected. The correct (optimal) dose is one which provides good tissue isolation but does not adversely affect tissue quality.

In ablating tissue, certain select electrodes 150 are activated as opposed to activating all of the electrodes 150. Only those electrodes 150 that are in direct contact with tissue are activated to deliver energy and form the tissue lesion.

Depending upon the visualization information, the basket 130 may need to be moved axially and/or rotationally in order to perform the ablation. For example, if the electrode spacing is too great, energy is delivered to form a first lesion segment and then the basket may need to be moved relative to the balloon (axially and/or rotationally) to reposition the electrodes and deliver energy to form a second lesion segment that is combined with the first lesion segment to form a more complete lesion segment. Alternatively, circumferential electrode spacing can be inferred from the endoscopic view and the PFA dosing adjusted to compensate for differing electrode spacing.

The shape and size of the formed lesion segment will depend on which electrodes were actuated and their locations. For example, activation of two electrodes 150 located along the same spline 140 will result in a formed lesion that extends more longitudinally, while activation of two electrodes 150 located along adjacent splines results in a formed lesion that extends more in a circumferential direction.

### Dual Transeptal/Secondary Catheter

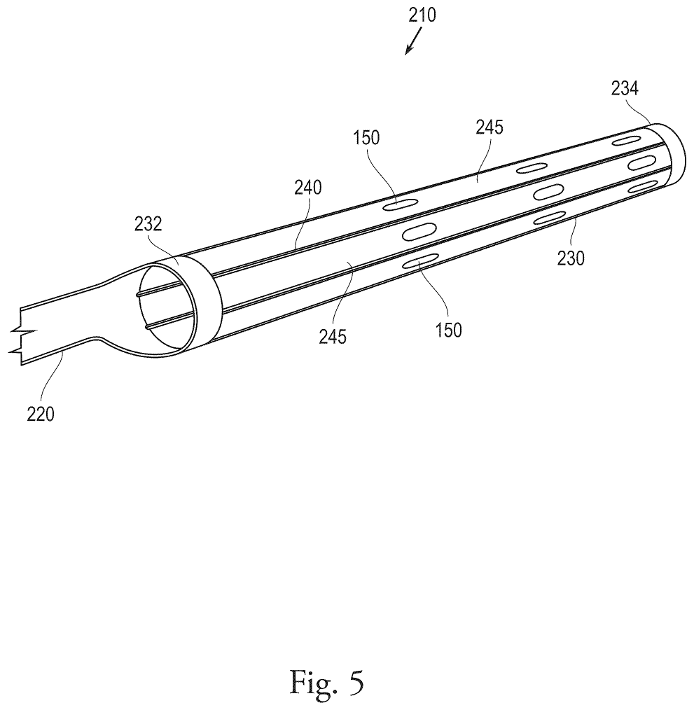

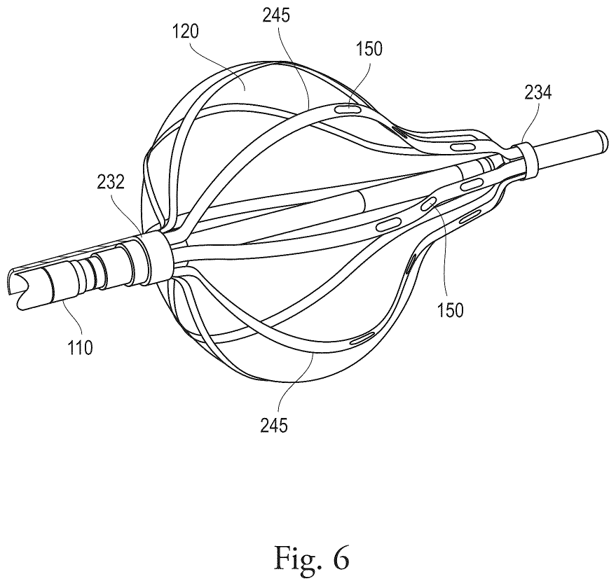

FIGS. 5 and 6 illustrate a balloon catheter 200 that is similar to the balloon catheter 100 with the exception that the balloon catheter 200 does not include the expandable basket 130. As a result, the reference numbers used in FIG. 4 are also used in FIGS. 5 and 6 for the parts that are in common to the two embodiments. The inflatable balloon is typically transparent and thus FIG. 6 shows the transparent nature of the balloon.

The balloon catheter 200 includes the main catheter shaft 110 that typically includes more than one shaft and often includes an inner catheter shaft and an outer catheter shaft or can otherwise include multiple concentric tubular structures as shown. The inflatable compliant balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

In this embodiment, there is a second catheter, namely, an electrode catheter 210 that is for use with the balloon catheter 200. The electrode catheter 210 comprises an elongated structure that has an open distal end and has a proximal region 220 and a distal electrode region 230. The proximal region 220 can comprise an elongated arcuate shaped body that is not completely circumferential in shape. Conversely, the distal electrode region 230 can be a completely circumferential structure. The distal electrode region 230 includes a proximal collar 232 at a proximal end of the distal electrode region 230 and a distal collar 234 at a distal end of the distal electrode region 230. Between the two collars 232, 234, the body of the distal electrode region 230 includes a plurality of longitudinal slits 240 that are spaced circumferentially about the body. These slits 240 define a plurality of longitudinal splines 245. The slits 240 do not extend into the areas of the two collars 232, 234. Much like the splines 140, the splines 245 carry one or more and preferably a plurality of the electrodes (e.g., electrodes 150) that are located along the outer surface (outer face) of the splines 245. Much like the previous embodiment, each spline 245 can carry a plurality of electrodes, such as three of more electrodes that are disposed in series and spaced apart from one another in the longitudinal direction of the spline 245.

Both ends of the distal electrode region 230 are open and thus, it represents an open ended tubular structure that, as described herein, is configured to receive the balloon catheter in its contracted (deflated) at rest state.

As in the previous embodiment, the splines 245 are not elastic and thus do not stretch but can expand in response to the expansion of the inflatable balloon 120. Therefore, the distance between the electrodes along the same spline 245 do not change based on whether the spline 245 is expanded or retracted. However, as in the previous embodiment, the distance between two electrodes on two different splines 245 does change based on the degree of expansion.

The balloon catheter is inserted into and through the hollow interior (inner lumen) of the electrode catheter 210 such that the splines 245 surround the inflatable balloon 130. As the balloon inflates, the splines 245 expand radially outward and separate from one another.

As in the other embodiment, the splines 245 can collapse as by retracting the splines 245 inside a main (outer) catheter shaft.

Visualization is used in this embodiment also to determine which electrodes are in contact with the tissue and also the visualization can guide the user in terms of making any adjustments with the balloon catheter and/or the electrode catheter in order to form a complete continuous lesion.

### Retractable Tines Electrode Array

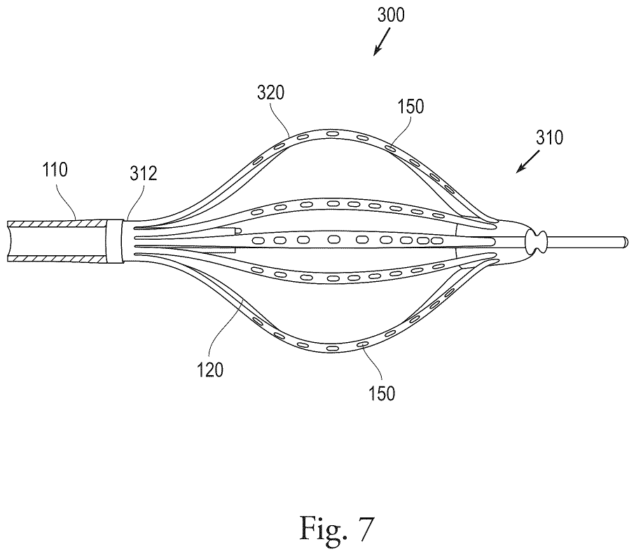

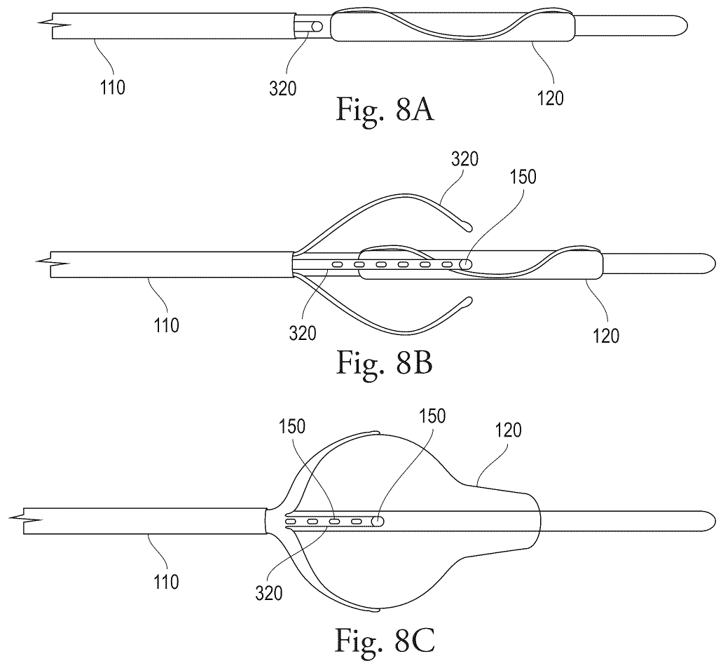

FIGS. 7 and 8A-C illustrate a balloon catheter 300 that is similar to the balloon catheter 100 with the exception that the balloon catheter 300 does not include the expandable basket 130. As a result, the reference numbers used in FIG. 4 are also used in FIGS. 7 and 8A-C for the parts that are in common to the two embodiments.

The balloon catheter 300 includes the main catheter shaft 110 that typically includes more than one shaft and often includes an inner catheter shaft and an outer catheter shaft or can otherwise include multiple concentric tubular structures as shown. The inflatable balloon 120 is included and is coupled to the main catheter shaft 110 and/or an additional shaft with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

The balloon catheter 300 further includes a retractable electrode sheath 310 that is configured to retract within the main catheter shaft 110 or another shaft of the catheter. Thus, as described herein, the retractable electrode sheath 310 is designed to move longitudinally along the main catheter shaft 110 and more particularly, the retractable electrode sheath 310 can travel within the main catheter shaft 110 to allow the retractable electrode sheath 310 to move between a fully retracted position and a fully extended position. In the fully retracted position, at least a substantial length of the retractable electrode sheath 310 is contained within the main catheter shaft 110 and in the fully extended position, a substantial length of the retractable electrode sheath 310 is disposed outside of the main catheter shaft 110 and surrounds the inflatable balloon 120 as described herein. As shown, in the fully extended position, the tines 320 can extend at least 75% of the length of the balloon 130 and can extend over 90% of the length of the balloon. In another embodiment, the tines 320 extend at least 50% of the length of the balloon 130 (e.g., they extend at least to the widest part of the inflated balloon 130).

The retractable electrode sheath 310 includes a proximal collar 312 that can be a continuous cylindrical structure and a plurality of expandable tines 320 that are integral at their proximal ends to the proximal collar 312. The tines 320 are cantilevered structures in that a distal end of each tine 320 is a free end and is not attached to another structure. The tines 320 are spaced apart and extend circumferentially around the balloon 130 when in the fully extended positions.

As in the other embodiments, the tines 320 are not elastic and do not stretch in any way; however, the tines 320 are able to expand (radially) outward as the inflatable balloon 130 inflates and similarly, when the inflatable balloon 130 deflates, the tines 320 can contract. The tines 320 thus can conform to the compliant balloon 130.

To cause retraction and full collapse of the tines 320, the retractable electrode sheath 310 is pulled back in the proximal direction and as the retractable electrode sheath 310 enters into the main catheter shaft 110, the presence of the main catheter shaft 110 in surrounding manner, applies an inward force to the tines 320 that collapses them and allows them to travel within the main catheter shaft 110 and retract away from the balloon 130.

As shown in the figures, each tine 320 includes one or more electrodes 150 and preferably a plurality of electrodes 150 that are spaced along the tine 320. The electrodes 150 are disposed in series along the length of the tine 320. The electrodes 150 along the tine 320 can be of the same type (e.g., same shape and size, etc.) or different type electrodes can be used in another embodiment.

As in the other embodiments, visualization (e.g., the endoscope) is used to determine which electrodes 150 are in contact with the tissue and those select electrodes can then be activated (actuated) to form the lesion. The user interface allows for the identification and powering of those electrodes 150 that are in contact with the tissue. As mentioned previously, the operating software can be programmed so that based on the distance between the activated electrodes 150, the proper dosing amount can be calculated and the requisite energy can be delivered to the electrodes 150.

As in all embodiments, it is desirable to limit the activation of electrodes to only those that are required to form the lesion (segment).

FIG. 8A shows the inflatable balloon 130 in a deflated state and the tines 320 are fully retracted and are located substantially within the main catheter shaft 110 (e.g., only the tips of the tines 320 protrude outside of the main catheter shaft 110).

FIG. 8B shows the inflatable balloon 130 still in its deflated state but the tines 320 have been deployed. As mentioned, the degree of coverage of the tines 320 relative to the balloon 130 can vary.

FIG. 8C shows the balloon 130 inflated and this results in the expansion of the deployed tines 320. In this figures, the tines 320 are shown extended about 50% the length of the balloon 130; however, this is merely exemplary in nature and it will be understood that it can extend along more or less of the balloon length.

The embodiment of FIGS. 7 and 8A-C thus consists of semi-rigid retractable tines 320 with one or more electrodes 150 along the outer surface of each tine 320, that are housed within the catheter (main catheter shaft 110) and deployed before inflating the balloon 130 (by sliding the retractable electrode sheath 310 distally using a controller or the like (manual or motorized). When the balloon 130 is inflated, the electrodes 150 are pressed against the inner surface of the vessel to achieve tissue contact. As with the other embodiments, the endoscope in this embodiment within the balloon 130 to allow for confirmation of tissue contact and electrode spacing under direct visualization, once tissue contact and desired electrode spacing is confirmed, energy is applied to the desired (selected) electrodes 150 to create the lesion. This embodiment can incorporate as few as four deployable tines 320, but a larger number of tines 320 is likely to provide the user with the ideal number of electrodes 150 and electrode spacing for effective treatment.

In this embodiment, as in the other embodiments, markers can be provided along the inner surface of the tine 320 to identify the location of the electrodes 150 along the tine 320 under visualization. This allows the user to determine which electrodes 150 are in contact with the tissue and then instruct the energy delivery module to deliver energy to those selected electrodes 150. In addition, in one embodiment, the system can include image recognition software that analyses the live image feed from the endoscope and identifies the electrode markers that are present. For example, if the markers, such as A1 and A2, are present, then the image recognition module will identify these electrodes and provide the user with the option to confirm that the electrodes that correspond to markers A1 and A2 should be activated and energy delivered to the user.

This image recognition functionality can be implemented in any of the other embodiments described herein in which the electrode markers are present to provide the user with a suggested electrode activation plan.

Balloon with PFA Braided Wire Mesh Electrode Array

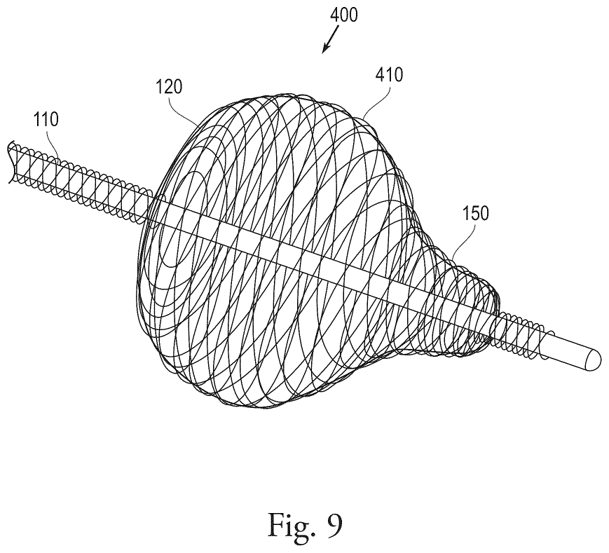

FIG. 9 illustrates a balloon catheter 400 that is similar to the other balloon catheters described herein. As a result, the reference numbers used in FIG. 4 are also used in FIG. 9 for the parts that are in common to the two embodiments.

The balloon catheter 400 includes the main catheter shaft 110 that typically includes more than one shaft and often includes an inner catheter shaft and an outer catheter shaft or can otherwise include multiple concentric tubular structures. The inflatable balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

The balloon catheter 400 includes a wire braid 410 that is disposed over the inflatable balloon 120 and is configured to expand radially as the inflatable balloon 120 is inflated. The wire braid 410 can comprise a mesh wire braid as shown. This wire mesh can be used as a support structure for an electrode array formed of electrodes 150 and can be formed of an insulating material. The electrodes 150 are disposed along the outer surface of the wire braid 410 and the coverage of the electrodes 150 can be uniform of non-uniform. In the non-uniform embodiment, the electrodes 150 can be more concentrated in one or more regions of the wire braid 410. For example, the electrodes 150 can be primarily located at the center region of the wire braid 410 where tissue contact is more likely.

In addition, the spacing between electrodes can be the same along the entire electrode array or the spacing can be different in one or more regions of the wire braid 410. For example, the spacing can be closer together in a central region of the wire braid 410.

As in the other embodiments, the electrodes 150 are connected to the energy source using conventional electric traces or wires (conductive paths) that are associated with and/or incorporated into the wire braid.

Alternatively, the wire braid 410 (support structure) itself can serve as and define the electrode array by incorporating an insulating coating on a conductive (metal) braid wires that is stripped off at desired locations for energy delivery by defining discrete electrodes in those area where the coating is removed. The wire braid 410 would be operatively connected to the energy source and electric current (energy) is delivered across the wire braid 410 with the areas in which the insulating coating is removed defining the electrodes that define the electrode array.

The wire mesh braid can be formed of separate discrete insulated wires to define discrete pathways along which the electrodes are present. By defining discrete electrode pathways, discrete regions of the wire mesh braid can be activated without activation of the other regions to allow for activation of those electrodes or that electrode region that is in contact with the tissue.

As shown, the wire braid 410 can extend beyond the inflatable balloon 130 in that one end of the wire braid 410 extends proximal to the inflatable balloon 130 and the other end of the wire braid 410 extends distal to the inflatable balloon 130.

As with the other embodiments, this embodiment once again uses an endoscope inside the balloon 130 in order to confirm electrode placement and tissue contact. The number of electrodes 150 in the array may vary along with the number of braid wires in order to achieve the most clinically effective energy delivery, and the user may be able to select or deselect a number of electrodes in order to customize the treatment zone.

Balloon with Embedded Electrode Array

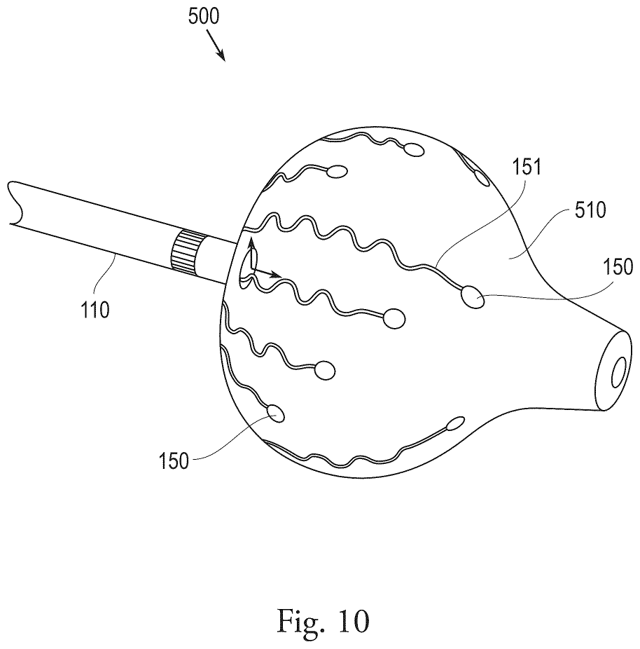

FIG. 10 illustrates a balloon catheter 500 that includes the main catheter shaft 110 along with an inflatable balloon 510 that is coupled to and extends along the main catheter shaft 110 as in the other embodiments. The distal end of the inflatable balloon 510 is coupled to a distal end of the main catheter shaft 110 and a proximal end of the inflatable balloon 510 is coupled to the main catheter shaft 110 at a location spaced from the distal end of the main catheter shaft 110.

The inflatable balloon 510 is a compliant balloon in which the electrodes 150 are integral. The balloon 510 itself includes electrodes 150 and flexible wire traces 151 embedded in the balloon material.

In this embodiment, the electrodes 150 can be disposed in and made integral to the balloon 510 as part of the molding process of the balloon 510. The electrodes 150 are spaced across the balloon 510 in a desired pattern. For example, the electrodes 150 are located circumferentially around the balloon 510. Alternatively, instead of being positioned and attached to the balloon material during the manufacture process, the electrodes 150 can be attached to the balloon 510 after the manufacture process. In particular, the electrodes 150 can be attached to the outer surface of the balloon 510 with the traces 151 also being attached to the outer surface of the balloon 510. Any number of conventional techniques can be used to attach these elements to the outside of the balloon 510 such as use of adhesive, bonding agents, etc.

The electrodes 150 are formed so that an outer surface of each electrode 150 is exposed along the surface of the balloon 150 for placement in contact with the tissue. Each flexible trace 151 is formed in a zig-zag pattern which is purposeful in order to permit the flexible traces 151 to move with the compliant balloon during inflation/deflation and during placement against the tissue. In other words, this zig-zag pattern accommodates the flexible traces 151 during the expansion and contraction of the balloon and prevents damage to the trace(s). Each flexible trace 151 is operatively coupled to the energy source so that energy can be delivered to select ones of the electrodes 150.

As with the other embodiments, this embodiment once again uses an endoscope inside the balloon 510 in order to confirm electrode placement and tissue contact. Once the user determines which electrodes 150 are in contact with the tissue, the user can then select these electrodes for activation.

In addition, electrode markers can be provided as in the other embodiments that are visible on the inside of the balloon 510 to the endoscope to allow the user or to allow image recognition software to determine which electrodes are clearly visible in the field of view of the endoscope. Based on this information, energy is delivered to those select electrodes 150 for forming the lesion. The user interface can be configured to easily allow the user the ability to select which electrodes to deliver energy to as by presenting the user with a touch screen with an electrode map and/or having the image recognition software prepopulate the screen with a proposed electrode activation map indicating which electrodes are visible in the endoscope and in contact with tissue.

Balloon with Micropores and Inner Electrode Array

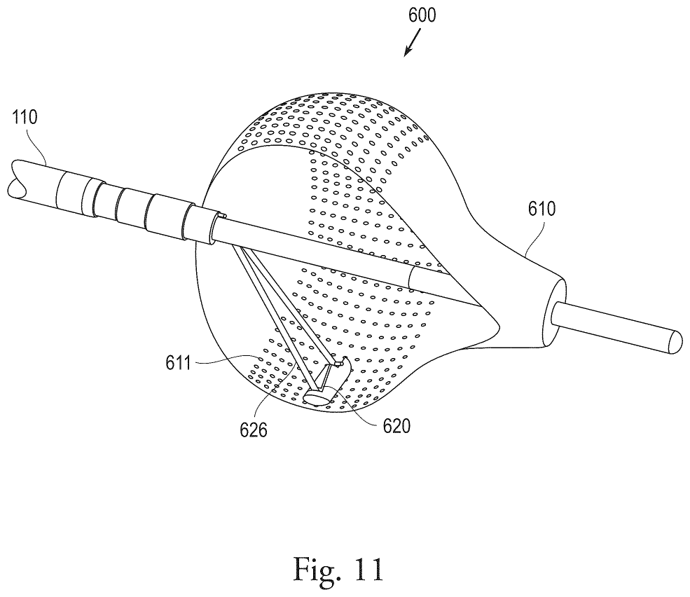

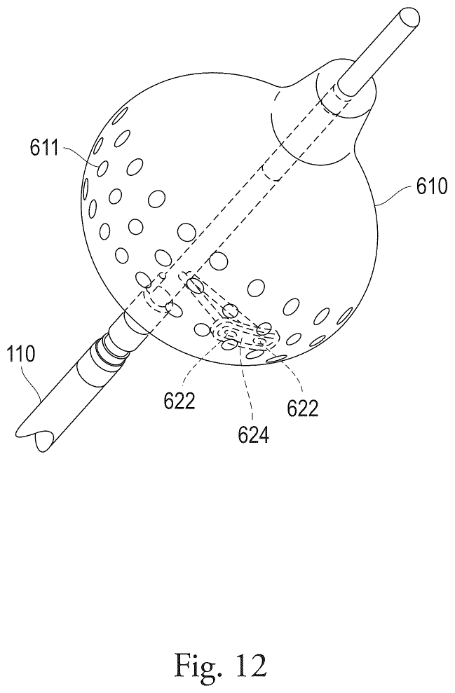

FIGS. 11 and 12 illustrate a balloon catheter 600 that includes the main catheter shaft 110 along with an inflatable compliant balloon 610 that is coupled to and extends along the main catheter shaft 110. An outer catheter body or sleeve 115 is also present and as mentioned, the catheter 600 can include other shafts, such as outer and inner catheter shafts, etc. The distal end of the inflatable balloon 610 is coupled to a distal end of the main catheter shaft 110 and a proximal end of the balloon 610 is coupled to the main catheter shaft 110 at a location that is spaced from the distal end.

As with the other embodiment, an endoscope is provided inside of the balloon 610 and can be coupled to the main catheter shaft 110. The endoscope is forward looking and allows view of the transparent balloon 610 and its contact with surrounding tissue.

In accordance with this embodiment, at least a portion of the balloon 610 has micropores 611 formed therein. The micropores 611 are preferably formed in one or more regions of the balloon 610 in which energy is to be delivered to the tissue. In the illustrated embodiment, the proximal and distal ends of the balloon 610 are devoid of micropores 611, while the center region includes the micropores 611 since it is this center region that contacts the tissue during use.

For ease of simplicity, the micropores 611 in FIG. 12 are shown as having greater dimensions than the micropores in FIG. 11; however, it will be understood that the micropores in FIGS. 11 and 12 can be the same size and the same number. However, FIG. 12 does convey that the micropores 611 can be formed to have different sizes and even different shapes.

The micropores 611 can have uniform constructions (i.e., same size and shape) or there can be two or more types of micropores 611. The micropores 611 can be formed in a uniform pattern as shown or can be formed in a non-uniform pattern. For example, as illustrated, the micropores 611 can be formed in a grid that extends circumferentially around the entire balloon 610.

The balloon catheter 600 also includes an electrode carrier 620 that is disposed within the balloon and can, in at least one embodiment, move within the balloon 610 (i.e., move rotationally within the balloon 610 and/or move longitudinally within the balloon 610). The electrode carrier 620 includes one or more electrodes 622 that are contained in a housing (hood) 624. In the illustrated embodiment, there is a pair of electrodes 622 in the housing 624 (however, it is possible to use a single electrode in the hood, with the hood rotating within the inside of the porous balloon). The housing 624 serves to contain and direct the energy of the electrodes 622. The electrodes 622 are placed in close proximity to the balloon itself, and the housing itself is placed in direct contact with the balloon's inner surface. The hood 624 can optimize the fraction of ablative energy delivered to tissue; however, the hood 624 can be eliminated and is not necessary.

The electrode array 622 is thus contained in the housing 624 which also serves to encapsulate a conductive liquid media, such as saline (e.g., normal saline or hypertonic saline) that allows for energy flow directly into the tissue via the micropores 611. In other words, the conductive liquid media can be delivered to the housing 624 as by use of one or more conduits 626 that open up into the inside of the housing 624. When the electrodes 622 are activated, energy is produced by the electrodes (e.g., between the electrodes) and since the electrodes 622 are bathed in the conductive liquid media, the energy serves to heat the conductive liquid media. The presence of the micropores 611 allows the heated conductive liquid media to weep through the micropores 611 to the tissue, which in combination with the energy from the electrodes 622 being conducted across the balloon material results in a target lesion being formed. In particular, a lesion segment is formed. To form a complete lesion, the electrode carrier 620 can be rotated and/or moved along the inner surface of the balloon. The electrode carrier 620 is held in contact with the inner surface of the balloon 610 by means of a mechanical adjustment controlled by the user, or a secondary balloon that can be inflated or deflated by the user to adjust the electrode contact pressure.

The combination of the electrode array and the conductive liquid media defines an electroconductive pathway used to form the lesion segment. It will be appreciated that the inflation media to control inflation or deflation of the balloon 610 can be the same or different than the conductive liquid media delivered to the inside of the housing 624.

In yet another embodiment, the balloon 610 does not include micropores 611 but instead is formed of a conductive balloon material (e.g., balloon material doped with carbon nanotubes). In this alternative embodiment, the housing (hood) can also be eliminated or it can be maintained. A non-conductive fluid can thus be used inside the balloon. The electrode array (or single electrode) is still disposed inside of the balloon 610 and is movable therein as by being able to freely rotate within the balloon and/or move longitudinally. Energy delivered to the electrode array is thus transferred to a local region of the conductive balloon that is in close proximity to the electrode array to form the lesion. In other words, the electrode array faces a localized area of the balloon and energy that is delivered to the electrode array is conducted to this localized area of the balloon to form the lesion.

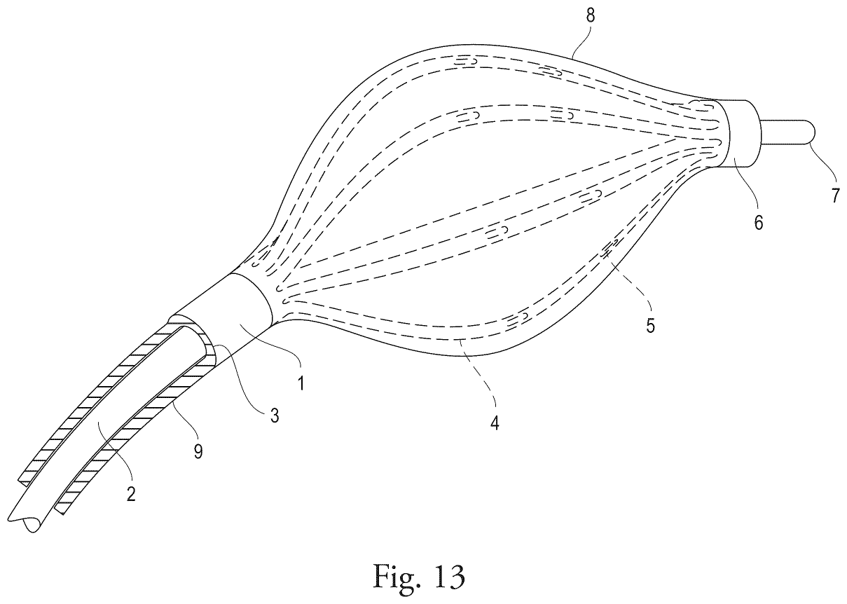

Now referring to FIG. 13, in yet another embodiment, a porous balloon catheter 700 is shown. The porous balloon catheter 700 is similar to the balloon catheter 600 and therefore, like elements are numbered alike. The balloon thus includes micropores 611. Instead of the electrode carrier 620, the balloon catheter 700 includes an elongate structure 710 that can be similar to the elongate structure 1 of FIG. 1 with several notable differences being that the elongate structure 710 is located inside the balloon as opposed to being located outside the balloon as in FIG. 1. The elongate structure 710 comprises a first tubular shaped portion 712 and a second tubular shaped portion 714 that surround the catheter shaft. The elongate structure 710 multifurcates into two or more but preferably six or more branches 720, with each branch 720 containing one or more electrodes 715 on their outward facing surface. The elongate structure 710 can be made of an elastic material pre-shaped into a geometry that allows it to expand and remain in contact with the balloon inner surface as the balloon is inflated. The elongate structure 710 would be collapsed by the balloon when the balloon is deflated by removing liquid from the balloon under vacuum. In other words, as the balloon is inflated, the elongate structure is constructed to automatically and naturally expand and similarly, it contracts due to the contraction of the balloon. This can naturally occur due to the memory characteristics of the elongate structure 710. The electrodes 715 that are on the outer surface of the elongate structure 710 are thus in contact with the inner surface of the porous balloon. As in the other embodiment, the balloon contains conductive fluid that passes through the micropores. Thus, energy from the electrodes 715 is conducted across the balloon itself and/or the conductive fluid within the balloon passes through the micropores to the target tissue.

It will be appreciated that in all embodiments, the electrodes are connected to a controllable energy source using conventional techniques, including electric leads, wires, conductive pathways, etc. The energy source can be controlled using traditional controls such as a master controller that can be a part of a console at which the user enters input and can control and select different operating parameters such as dosing information (dose power (wattage), etc.

Those embodiments that incorporate an electrode array are particularly suited for delivery of electroporative ablation energy (PFA).

Additional details concerning certain embodiments of the present disclosure are as follows.

A device for the alteration of tissue for the purpose of changing, amongst other things, the conducting properties of the tissue to achieve a desired result.

An external sheath that is positioned over the existing catheter system.

Consisting of three distinct portions, a location collar of a hard material at the most distal end, a balloon expandable section of softer, more pliable material (or alternative arrangement) located in the vicinity of the primary balloon and an overcoat on the body of the catheter continuing until near the proximal end.

Electrodes may be placed on the hard collar section for measurement of distal electrical activity or may be employed in the delivery of energy.

Electrodes are primarily placed on the balloon expandable section, for the delivery of energy to achieve the alteration of the properties of the target tissue, in a variety of configurations (another section)

The overcoat of the body incorporates the conductors for the distal measurement and energy delivery and are terminated in the vicinity of the control for the rotation of the other energy delivery source.

The electrodes on the collar may be in a variety of configurations, including square electrodes in a 2, 4, or 6 style equally spaced around the measuring area on the collar.

The balloon expandable area electrodes are intended to be the primary energy delivery (therapeutic) of the device. The most likely embodiment would be 16 electrode arrangement, equally spaced, positioned proximally to the primary treatment area, allowing the balloon to be deflated slightly to allow the electrode array to be extended distally into the area to be treated, perhaps, but not necessarily in an arc similar in location to where the primary energy was or will be delivered. The area will be aligned so that at the inflation pressure designated for “PFA” therapy, the electrodes will be equally spaced and are separate, so that they may be accessed individually or in a variety of groupings.

The overcoat of the catheter will have the conducting means for all of the sensing and energy delivery electrodes (some or all of them serve dual purposes) so as not to provide any, or at least a minimal amount, of impingement on the flexure or rotation of the primary catheter. This may be a spiral routing with the ability to use a variety of spiral pitches.

The balloon expandable section of the device may be a complete sheath consisting of a very elastic material with the electrodes on the surface or may be more rigid with sections of the device removed so that the electrodes are placed into the desired area by displacement of the structure.

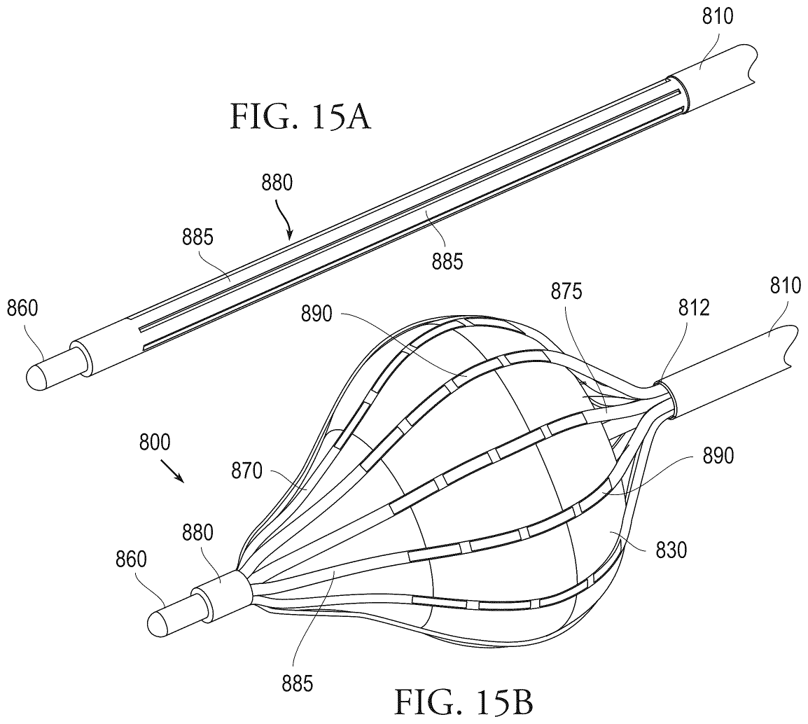

### Translating Tip Balloon Catheter (FIGS. 15A and 15B)

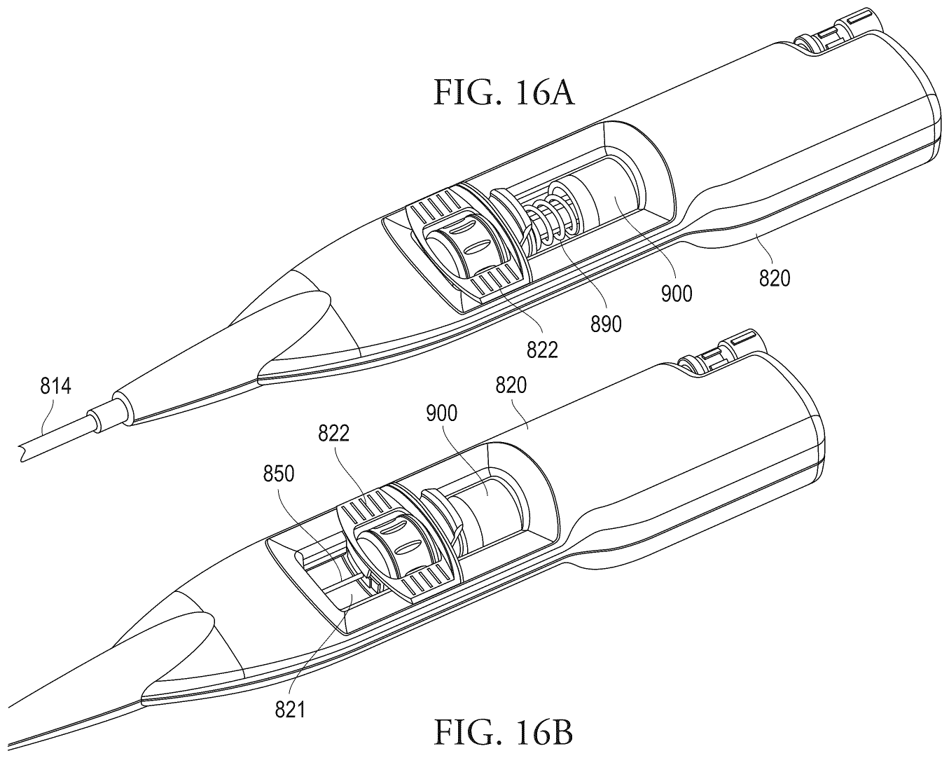

Now referring to FIGS. 15A and 15B in which a translating tip balloon catheter 800 is illustrated. FIG. 15A shows the catheter 800 in a collapsed state for delivery to the target site, while FIG. 15B shows the catheter 800 in an in use, expanded state. The catheter 800 includes an elongated outer catheter (shaft) 810 that has a distal end 812 and an opposite proximal end 814 (FIG. 16A). The outer catheter 810 is an elongated hollow structure. The catheter 800 also includes a handle 820 (FIG. 16A) for grasping by the user. The handle 820 is coupled to the proximal end 814 of the outer catheter 810. Additional details concerning the handle 820 are set forth below.

The handle 820 can take any number of different forms including being formed of two parts that are attached to one another to define the hollow interior that houses the working parts of the translating tip balloon catheter 800.

The catheter 800 also includes an inflatable balloon 830 that is coupled to the distal end 812 of the outer catheter 810. The outer catheter 810 can terminate proximally to the balloon 830 or it can extend partially into the balloon 830; however, the outer catheter 810 does not extend completely to the distal end of the catheter 800.

The balloon 830 comprises a compliant balloon. It will be appreciated that the catheter 800 and balloon 830 have traditional inflation and deflation architecture such as an inflation and/or deflation lumen through which inflation media flows into the balloon for inflation thereof. As is known, the inflation media can be circulated using a pump or the like.

The catheter 800 is configured to deliver PFA energy using an expandable electrode basket construction that surrounds the balloon 830 along with an actuator or translation mechanism that allows the electrode basket construction to both expand and collapse to a more flattened state. More specifically, the translation mechanism can include an elongated structure such as a tube or solid rod 850 (FIG. 16B) that is coupled at a first (proximal) end to the handle and is coupled at an opposite second (distal) end to a flexible nose tip 860. Thus, while the element 850 is described as being a tube, it will be appreciated that it does not have to have a tubular structure but can be solid.

In one embodiment, the tube 850 comprises a nitinol tube.

The nose tip 860 defines the distal end of the catheter 800. The nose tip 860 is not directly attached to the outer catheter 810 but instead is capable of axial movement thereto and this provides the axial translation aspect of the catheter 800. The distal end of the tube 850 is thus fixedly attached to the nose tip 860 and passes through the inside of the balloon 830. As a result, when the tube 850 is driven in a forward direction, the nose tip 860 is driven forward and conversely, when the tube 850 is driven in a rearward direction, the nose tip 860 is driven rearward towards the handle.

The expandable electrode basket, according to one embodiment, is formed of a first electrode basket 870 and a second electrode basket 880 that are described in more detail herein. Each of the first electrode basket 870 and the second electrode basket 880 is coupled to both the distal end 812 of the outer catheter 810 and to the nose tip 860. As illustrated, the first and second electrode baskets 870, 880 are layered in that the first electrode basket 870 can be considered to be an inner basket and the second electrode basket 880 can be considered to be an outer basket.

The first electrode basket 870 includes a distal end portion that can be in the form of a solid cylindrical portion and a proximal end portion that can also be in the form of a solid cylindrical portion. Similarly, the second electrode basket 880 includes a distal end portion that can be in the form of a solid cylindrical portion and a proximal end portion that can also be in the form of a solid cylindrical portion. The distal end portion of the first electrode basket 870 is coupled to and can surround the nose tip 860, while the distal end portion (e.g., a collar) of the second electrode basket 880 can be disposed directly over the distal end portion (e.g., a collar) of the first electrode basket 870 and thus surrounds (superimposed over) the distal end portion of the first electrode basket 870. Similarly, the proximal end portion (e.g., a collar) of the second electrode basket 880 can be disposed over the proximal end portion (e.g., a collar) of the first electrode basket 870 and thus surrounds (superimposed over) the proximal end portion of the first electrode basket 870. In other words, the first electrode basket can be a slitted tubular structure with solid ends and the second electrode basket can be a slitted tubular structure with solid ends. These two tubular structures are superimposed with the slitted tubular structure of the second electrode basket being disposed directly over the slitted tubular structure of the first electrode basket with the slits circumferentially offset from one another.

Each of the first electrode basket 870 and the second electrode basket 880 carries one or more electrodes. As illustrated, the first electrode basket 870 and the second electrode basket 880 are splined structures in that the first electrode basket 870 includes a plurality of longitudinal slits that create and define a plurality of first splines 875 that extend circumferentially around the first electrode basket 870. The second electrode basket 880 includes a plurality of longitudinal slits that create and define a plurality of second splines 885 that extend circumferentially around the second electrode basket 880. As described herein, the locations of the first and second splines 875, 885 are purposely selected by orientation of the first and second electrode baskets 870, 800 such that when the balloon 830 expands and the first and second splines 875, 885 move to their expanded states (FIG. 15B), the first and second splines 875, 885 do not overlap but are interposed within one another. In other words, each second spline 885 is located between two adjacent first splines 885 and vice versa. This action allows complete circumferential coverage around the balloon 830.

In one embodiment, each of the first and second electrode baskets 870, 880 are constructed such that they have six (6) splines and as a result, the superimposed and offset nature of the first and second electrode baskets 870, 880 defines twelve (12) splines. The first and second electrode baskets 870, 880 can thus have the same or very similar structure with one fitted over the other and rotated so as to rotationally offset the splines of each. Since each spline can carry one or more electrodes, when the balloon is inflated and the splines 875, 885 are in the expanded state, the twelve splines are circumferentially spaced along the outside of the balloon and provide increased electrode coverage for contact with tissue. If only one electrode basket (one sheath or collar) was used, the inclusion of enough longitudinal slits to form twelve splines would result in each spline having insufficient width to carry the desired ablation electrodes (e.g., for PFA). The present arrangement of two superimposed electrode baskets that are rotationally offset overcomes this deficiency and permits twelve splines each of sufficient width to carry one or more electrodes of desired size suitable for PFA.

In the initial, collapsed state, the first splines 875 are at least substantially covered by the second electrode basket 880 and thus, in this initial, collapsed state, only the second electrode basket 880 is substantially visible since the first electrode basket 880 is covered.

In one embodiment, each spline 875, 885 carries one or more electrodes 890 (FIG. 15A omits the electrodes 890 for case of illustration). In the illustrated embodiment, each spline 875, 885 includes four electrodes 890 that are spaced apart and arranged in series (longitudinally) along the spline. In one embodiment, each electrode 890 can be controlled independently or in another embodiment, all of the electrodes 890 on one spline 875, 885 can be controlled together. As is known, a controller is used to control the supply of the ablation energy (e.g., PFA) to the electrodes 890 and in some embodiments, visualization can be used to detect which spline electrodes 890 are in contact with the target tissue. In one embodiment, instead of applying energy to all electrodes 890, energy can be supplied to select spline electrodes 890 (e.g., those electrodes 890 in contact with the target tissue).

The first and second electrode baskets 870, 880 are not compliant like the balloon 830. In one embodiment, the first and second electrode baskets 870, 880 are formed of polyimide.

The electrodes 890 are secured to the splines using conventional techniques, such as bonding, etc. Each electrode 890 can be connected to the energy source with an electrical trace as is known. For example, the electrical traces can be copper traces and the electrodes 890 can be gold plated electrodes.

Now referring to FIGS. 15A-16B, the handle 820 includes a through opening 821 in which an actuator 822 is movable disposed and contained. For example, the actuator 822 can be in the form of a slider that can be accessed and manipulated one opposite sides (faces) the handle 820. A forward end of the actuator 822 is fixedly coupled to the tube 850 and therefore, when the actuator 822 is moved axially, the tube 850 moves axially since they are fixed to one another (no relative movement therebetween). Thus, when the actuator 822 is moved forward, the tube 850 moves forward and this is translated into the nose tip 860 moving forward. Since the balloon 830 and each of the first and second electrode baskets 870, 880 are fixedly attached to the nose tip 860, all of these structure move forward as well. The forward movement results in flattening (elongation) of the balloon 830 and the first and second electrode baskets 870, 880 toward and to the position shown in FIG. 15A. Conversely, when the actuator 822 is moved rearward, the tube 850 moves rearward and this is translated into the nose tip 860 moving rearward. Since the balloon 830 and each of the first and second electrode baskets 870, 880 are fixedly attached to the nose tip 860, all of these structure move rearward as well. The rearward movement results in expansion (radially outward movement) of the balloon 830 and the first and second electrode baskets 870, 880 toward and to the position shown in FIG. 15B.

When the balloon 830 inflates, it expands the splines 875, 885 to the balloon shape, which pulls this nose tip 860 in a proximal direction.

Additional details concerning the actuator 822 are found in U.S. Pat. No. 11,389,236, which is hereby incorporated by reference in its entirety.

In yet another aspect, the handle 820 includes a biasing mechanism that operates on the tube 850. In particular, the biasing mechanism can be in the form of a spring 890 that is contained within the handle and applies a biasing force to the rear of the actuator 822. The spring 890 can be a coil spring. One end of the spring 890 seats against a fixed surface of the handle, while the opposite end seats against the rear of the actuator 822. The spring 890 shown is designed to assist in some of these movements. To deliver the catheter 800 to the target location, the catheter 800 can be delivered through a delivery sheath (not shown). Going through the sheath, the distal nose tip 860 will be pushed in a proximal direction by the frictional forces of going into the sheath. Some force will be required to resist this movement, and the spring 890 would provide that force. Once inside the patient and in place, the doctor will inflate the balloon 830.

In one implementation of this design, the spring 890 is designed such that the inflation of the balloon 830 is enough to overcome the spring 890 and pull the distal nose tip 860 backwards, allowing the splines 875, 885 to expand to the shape of the balloon 830. In another implementation, there will be some sort of lock that can be removed so that the spring 890 is either not pushing on the nitinol tube 850 or is pushing on it less. This is shown simply for now by a removable block 900 proximal to the spring 890.

FIG. 16A shows the handle and the actuator 822 in an extended state and FIG. 16B is a perspective view of the handle of FIG. 16A in a retracted state. When the balloon 830 is deflated and the first and second electrode baskets 870, 880 are in the flattened state of FIG. 15A, the actuator 822 is in the position shown in FIG. 16A and conversely, when the balloon 830 is the inflated state of FIG. 15B, the actuator 822 is in the position shown in FIG. 16B. Thus, as the balloon 830 is deflated, at some point, the spring force of the spring 890 overcomes the applied force of the balloon 830 and the actuator 822 is driven to the extended position shown in FIG. 16A which is the rest position of the catheter when the balloon 830 is deflated and the first and second electrode baskets 870, 880 are in the flattened state of FIG. 15A.

It will also be appreciated that in another embodiment, the catheter 800 only includes a single electrode basket, namely the first electrode basket 870 with a plurality of first splines 875. The second electrode basket 870 is thus eliminated. In one embodiment, the plurality of first splines 875 comprises 6 or more splines that are circumferentially spaced apart. Thus, the single basket embodiment will appear as shown in FIG. 15A in that there is one electrode basket coupled to the outer catheter 810 and to the nose tip 860 as described herein. It will be understood that in FIG. 15A, the outermost second electrode basket 880 is shown; however, in a single basket design, the second electrode basket of FIG. 15A would represent the single (only) electrode basket that can be described as being the first electrode basket since there is only one.

The single catheter basket design works exactly like the two basket design in that the user manipulates the actuator 822 to cause axial movement of the nitinol tube 850 which results in axial movement of the nose tip 860 resulting in the single electrode basket moving between the flattened state (FIG. 15A) and the expanded state, like FIG. 15B, when the balloon 830 inflates.

In the single catheter basket design, the handle can be spring loaded as well as described herein.

It will be appreciated that in one embodiment, the number of splines can be more than two and the widths of the splines are sufficient to support the one or more electrodes that are disposed on one or more electrodes. As with the other embodiment(s), each spline 875 can include one or more electrodes spaced longitudinally therealong for tissue ablation (e.g., PFA).

The nitinol tube 850 serves as an axial push/pull rod in each of these embodiments whether there is one or two electrode baskets 870, 880 surrounding the balloon 830. The movement of the nitinol tube 850 is preferably controlled within the handle.

It will also be understood that within the balloon 830, there can be a visualization device, such as the endoscope described herein. Such visualization device assists the user in determination of the degree of contact and location of the contact between the balloon and tissue (as opposed to the presence of a blood pool).

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. An ablation balloon catheter comprising:
an outer catheter shaft;
an inflatable balloon coupled at a first end to the outer catheter shaft;
a translatable nose tip to which a second end of the inflatable balloon is coupled;
a first electrode basket having a plurality of first splines that surround the balloon, the first electrode basket being coupled at a first end to the outer catheter shaft and at a second end to the nose tip; and
a second electrode basket having a plurality of second splines that surround the balloon, the second electrode basket being coupled at a first end to the outer catheter shaft and at a second end to the nose tip, wherein the second electrode basket is disposed over the first electrode basket and the plurality of first splines are rotationally offset from the plurality of second splines, wherein one or more of the first splines support one or more electrodes and one or more of the second splines support one or more electrodes;
an actuator for axially translating the nose tip to facilitate the first and second electrode baskets moving to a collapsed state when the balloon is deflated;
wherein the plurality of first splines and the plurality of second splines are configured to deploy and radially expand under inflation of the inflatable balloon with the plurality of first splines interposed between the plurality of second splines.

2. The ablation balloon catheter of claim 1, wherein the plurality of first splines comprises six splines and wherein the plurality of second splines comprises six splines.

3. The ablation balloon catheter of claim 1, wherein a distal end and a proximal end of each of the first electrode basket and the second electrode basket comprises a solid cylindrical shaped collar body, the distal end of the second electrode basket covering the distal end of the first electrode basket in both the collapsed state and an expanded state of the first and second splines.

4. The ablation balloon catheter of claim 1, wherein each first spline and each second spline has at least one electrode disposed on an outer surface thereof.

5. The ablation balloon catheter of claim 4, wherein each first spline and each second spline has a plurality of electrodes disposed on an outer surface thereof in spaced relationship.

6. The ablation balloon catheter of claim 1, wherein the balloon comprises a compliant balloon and the first and second electrode baskets are formed of a non-compliant material.

7. The ablation balloon catheter of claim 6, wherein the first and second electrode baskets are formed of polyimide.

8. The ablation balloon catheter of claim 1, wherein the actuator comprises an elongated structure that is coupled at a first end to an axially movable part contained in the handle and is coupled at an opposite second end to the nose tip.

9. The ablation balloon catheter of claim 8, wherein the elongated structure comprises a tube.

10. The ablation balloon catheter of claim 9, wherein the tube comprises a nitinol tube.

11. The ablation balloon catheter of claim 1, wherein the nose tip comprises a flexible blunt end tip.

12. The ablation balloon catheter of claim 8, wherein the movable part of the handle comprises a slider contained within handle and being movable in an axial direction between an extended position and a retracted position.

13. The ablation balloon catheter of claim 12, wherein the slider is in the extended position when the balloon is deflated and the first and second splines are in the collapsed states and is in the retracted position when the balloon is inflated and the first and second splines are in the expanded states.

14. The ablation balloon catheter of claim 8, wherein the movable part is spring biased.

15. The ablation balloon catheter of claim 12, wherein the slider is spring biased and the movable part assumes the extended position as an at rest position of the movable part and assumes the retracted position when the balloon is inflated and force of the balloon on the first and second splines overcomes a spring force of the spring.

16. The ablation balloon catheter of claim 1, further including a visualization device that is disposed within the balloon.

17. The ablation balloon catheter of claim 16, wherein the visualization device comprises a movable endoscope.

18. The ablation balloon catheter of claim 17, wherein the endoscope is rotatable and can move axially within the balloon.

19. The ablation balloon catheter of claim 1, wherein the actuator comprises an elongated nitinol tube that is coupled at a first end to an axially movable part contained in the handle and is coupled at an opposite second end to the nose tip, the tube passing through the balloon.

20. The ablation balloon catheter of claim 1, wherein each electrode includes a conductive trace that is operatively coupled to an ablative energy source.

21. The ablation balloon catheter of claim 20, wherein the ablative energy source comprises PFA.

22. The ablation balloon catheter of claim 1, wherein a distal end and a proximal end of each of the first electrode basket and the second electrode basket comprises a solid cylindrical shaped collar body, the distal end of the second electrode basket covering the distal end of the first electrode basket in both the collapsed state and an expanded state of the first and second splines, wherein the solid cylindrical shaped collar body at the proximal ends is at least partially disposed under the outer catheter shaft.

23. An ablation balloon catheter comprising:
an outer catheter shaft;
an inflatable balloon coupled at a first end to the outer catheter shaft;
an axially translatable nose tip to which a second end of the inflatable balloon is coupled;
an electrode basket surrounding the balloon, the electrode basket having a plurality of first splines and a plurality of second splines, the electrode basket being coupled to the outer catheter shaft and to the nose tip, the electrode basket moving between a flattened state and an expanded state; and
an actuator for axially translating the nose tip to facilitate the first and second electrode baskets moving to a collapsed state when the balloon is deflated, the actuator moving axially between an extended position and a retracted position;
wherein the plurality of first splines and the plurality of second splines are configured to deploy and radially expand under inflation of the inflatable balloon;
wherein the actuator is spring biased and assumes the extended position as an at rest position and assumes the retracted position when the balloon is inflated and force of the balloon on the plurality of first and second splines overcomes a spring force of the spring.

24. An ablation balloon catheter comprising:
an outer catheter shaft;
an inflatable balloon coupled at a first end to the outer catheter shaft;
a forward-looking endoscope disposed within an interior of the inflatable balloon;
a translatable nose tip to which a second end of the inflatable balloon is coupled;
a first electrode basket having a plurality of first splines that surround an exterior of the balloon, the first electrode basket being coupled at a first end to the outer catheter shaft and at a second end to the nose tip, wherein one or more of the first splines support one or more electrodes;
an actuator for axially translating the nose tip to facilitate the first electrode basket moving to a collapsed state when the balloon is deflated;
wherein the plurality of first splines are configured to deploy and radially expand under inflation of the inflatable balloon.

25. The ablation balloon catheter of claim 24, further comprising a second electrode basket having a plurality of second splines, the second electrode basket being coupled at a first end to the outer catheter shaft and at a second end to the nose tip, wherein the second electrode basket is disposed over the first electrode basket and the plurality of first splines are rotationally offset from the plurality of second splines, wherein one or more of the second splines support one or more electrodes; and
wherein the plurality of second splines are configured to deploy and radially expand under inflation of the inflatable balloon.

26. The ablation balloon catheter of claim 24, wherein the actuator moves axially between an extended position and a retracted position; and
wherein the actuator is spring biased and assumes the extended position as an at rest position and assumes the retracted position when the balloon is inflated and force of the balloon on the plurality of first splines overcomes a spring force of the spring.
